(19) United States
(12) Patent Application Publication
(10) Pub. No.: US 2026/0110017 A1
(43) Pub. Date: Apr. 23, 2026

(54) SYSTEMS, METHODS, AND COMPOSITIONS FOR DIGITAL DROP-OFF ASSAYS

(71) Applicant: ChromaCode, Inc., Carlsbad, CA (US)

(72) Inventors: Lucien JACKY, Carlsbad, CA (US); Jerrod SCHWARTZ, Carlsbad, CA (US)

(21) Appl. No.: 18/994,478

(22) PCT Filed: Jul. 14, 2023
(86) PCT No.: PCT/US2023/070277

§ 371 Date: Jan. 14, 2025

Related U.S. Application Data
(60) Provisional application No. 63/368,536, filed on Jul. 15, 2022.
(60) Provisional application No. 63/378,603, filed on Oct. 6, 2022.

(51) Int. Cl. C12Q 1/6823

(52) U.S. Cl. CPC C12Q 1/6823

(57) ABSTRACT

The present disclosure provides methods, systems, and compositions for the multiplexed detection and quantification of multiple analytes from a sample. Analytes may be nucleic acid analytes. Detection of analytes may comprise contacting one or more samples with primers and/or hybridization probes to generate cumulative signal measurements. The methods may comprise digital PCR or may comprise partitioning a sample into multiple partitions.

### CROSS-REFERENCE

This application claims the benefit of priority to U.S. Provisional App. No. 63/368,536 filed Jul. 15, 2022, and U.S. Provisional App. No. 63/378,603 filed Oct. 6, 2022, which are incorporated by reference herein in its entirety.

### BACKGROUND OF THE INVENTION

Detection of nucleic acid sequences are used for a wide variety of purposes. Detection of a particular nucleic acid sequence in a gene of a subject may indicate that the subject may have a particular disorder or be more prone to having a particular disorder. Detection of nucleic acid sequences may be used to detect an infection by detecting a gene or nucleic acid sequence of a pathogen. PCR may be used to amplify nucleic acids for analysis.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### SUMMARY

Disclosed herein, in some aspects, are methods, systems and compositions for detection multiple analytes.

In an aspect, the present disclosure provides a method of identifying the presence of an analyte in a sample, the method comprising: a) providing to said sample a primer oligonucleotide comprising a first region, wherein the first region hybridizes to said analyte and a second region comprising two or more distinct probe binding sites; b) subjecting said primer oligonucleotide to an extension reaction, thereby generating a probe binding nucleic acid; c) annealing (i) a second primer oligonucleotide and (ii) one or more probes to said probe binding nucleic acid; d) subjecting said second primer oligonucleotide to an extension reaction thereby generating one or more signals; and e) identifying the presence of said analyte based at least on detection of an intensity level and a wavelength of said one or more signals.

In some embodiments, the one or more signals is detected in more than one channel. In some embodiments, the one or more signals is two or more signals. In some embodiments, the one or more signals is three or more signals. In some embodiments, the one or more signals is four or more signals. In some embodiments, the one or more signals is detected in more than two channels. In some embodiments, the one or more signals is detected in more than three channels. In some embodiments, a signal of said one or more signals is detected at an intensity level of 1i. In some embodiments, a signal of said one or more signals is detected at an intensity level of 2i. In some embodiments, the one or more signals is generated from more than one probe that binds to said probe binding nucleic acid. In some embodiments, the one or more signals is generated via degradation of one or more probes that binds to said probe binding nucleic acid. In some embodiments, a probe of said one or more probes comprises fluorophore. In some embodiments, the probe comprises a quencher. In some embodiments, the fluorophore is selected from a group consisting of FAM, HEX, TAMRA, Cy5, and combinations thereof. In some embodiments, the second region comprises three or more distinct probe binding sites. In some embodiments, the second region comprises four distinct probe binding sites. In some embodiments, the method comprises, prior to c), providing a first probe that binds to first probe binding site of said probe binding nucleic acid and a second probe that binds to a second probe binding site of said probe binding nucleic acid. In some embodiments, the first probe is present in a same concentration as a concentration of said second probe. In some embodiments, the first probe is present in a different concentration as a concentration of said second probe. In some embodiments, the first probe is present in a greater concentration than a concentration of said second probe. In some embodiments, the first probe is present in more than two times greater concentration than a concentration of said second probes. In some embodiments, a) further comprises providing a first reverse oligonucleotide that hybridizes to said analyte. In some embodiments, b) comprises performing an amplification reaction to generate a plurality of amplicons of said probe binding nucleic acid. In some embodiments, the method further comprises, subsequent to a) and prior to b) generating a plurality of partitions. In some embodiments, the analyte comprises a nucleic acid. In some embodiments, the nucleic acid comprises a DNA or RNA. In some embodiments, the first primer oligonucleotide further comprises a third region 5′ to said second and first region. In some embodiments, c) comprises annealing said second primer oligonucleotide to said third region. In some embodiments, c) comprises annealing said second primer oligonucleotide to at least a portion of said second region. In some embodiments, the method further comprises, identifying the presence of a second analyte, wherein a) comprises providing a third primer oligonucleotide comprising a first region that hybridizes to said second analyte and a second region comprising one or more probe binding sites. In some embodiments, the analyte is detected at a sensitivity of a least 95%. In some embodiments, the analyte is detected at a sensitivity of a least 99%.

In an aspect, the present disclosure provides a method of identifying the presence of one or more analytes of a plurality of analytes in a sample, the method comprising: a) providing to said sample a plurality of tailed primers wherein a first tailed primer of said plurality of tailed primers comprises a first region that hybridizes to an analyte and a second region comprising a first set of one or more probe binding sites, and a second tailed primer of said plurality of tailed primers comprises a first region that hybridizes to a second analyte and a second region comprising a second set of one or more probe binding sites, wherein said first set of probe binding sites and said second set of probe binding sites are not identical; b) subjecting said plurality of tailed primers to extension reactions, thereby generating probe binding nucleic acids; c) annealing a plurality of second primers to said probe binding nucleic acids; d) subjecting said plurality of second primers to extension reactions to generate (i) if the first analyte is present, a first set of one or more signals, and (ii) if the second analyte is present, a second set of one or more signals; and e) based at least on if present, said first set of one or more signals and, if present, said second set of one or more signals, identifying the presence of one or more analytes of the plurality of analytes. In some embodiments, d) comprises generating (i) if said first analyte is present, a first set of one or more signals from one or more probes that bind said first set of probe binding sites, and (ii) if said second analyte is present, a second set of one or more signals from one or more probes that bind said second set of probe binding sites. In some embodiments, the first set of one or more signals or second set of one or more signals is detected in more than one channel. In some embodiments, the first set of one or more signals or second set of one or more signals is detected in more than two channels. In some embodiments, the first set of one or more signals or second set of one or more signals is detected in more than three channels. In some embodiments, the first set of one or more signals or second set of one or more signals is detected in more than four channels. In some embodiments, the identifying the presence of the plurality of analytes is based at least on identifying an intensity level of the signals of the first or second set of one or more signals. In some embodiments, a signal of said first or second set of one or more signals is detected at an intensity level of 1i. In some embodiments, a signal of said first or second set of one or more signals is detected at an intensity level of 2i. In some embodiments, a first signal of the first set of one or more signals is detected in a first channel at an intensity of 1i and a second signal of the first set of one or more signals is detecting in a second channel at an intensity of 1i. In some embodiments, a first signal of the first set of one or more signals is detected in a first channel at an intensity of 1i and a second signal of the first set of one or more signals is detecting in a second channel at an intensity of 2i. In some embodiments, the first or second set of one or more signals is generated from more than one probe that binds to said probe binding nucleic acid. In some embodiments, the first or second set of one or more signals is generated via degradation of said a probe that binds to said probe binding nucleic acid. In some embodiments, the method further comprises, subsequent to b) and prior to d), annealing one or more probes to the first or second set of probe binding sites. In some embodiments, a probe of said one or more probes comprises fluorophore. In some embodiments, the probe comprises a quencher. In some embodiments, the fluorophore is selected from a group consisting of FAM, HEX, TAMRA, Cy5, and combinations thereof. In some embodiments, the second region of the first tailed primer or the second tailed primer comprises two or more probe binding sites. In some embodiments, the second region of the first tailed primer or the second tailed primer comprises three or more probe binding sites. In some embodiments, the second region of the first tailed primer or the second tailed primer comprises four probe binding sites. In some embodiments, the method comprises, prior to d), providing a first probe that binds to first probe binding site of said probe binding nucleic acids and a second probe that binds to a second probe binding site of said probe binding nucleic acids. In some embodiments, the first probe is present in a same concentration as a concentration of said second probe. In some embodiments, the first probe is present in a different concentration as a concentration of said second probe. In some embodiments, the first probe is present in a greater concentration than a concentration of said second probe. In some embodiments, the first probe is present in more than 2 times greater concentration than a concentration of said second probes. In some embodiments, a) further comprises providing a third oligonucleotide comprising a region substantially complementary to said analyte. In some embodiments, b) comprises performing an amplification reaction to generate a plurality of amplicons of said probe binding nucleic acids. In some embodiments, the method further comprises, subsequent to a) and prior to b) generating a plurality of partitions. In some embodiments, a first partition of said plurality of partition comprises said first analyte and a second partition of said plurality of partition comprises said second analytes. In some embodiments, the first set of one or more signals are generated in said first partition and said second set of signal and generated in said second partition. In some embodiments, the method further comprises, detecting said first set of one or more signals separately from detecting said second set of one or more signals. In some embodiments, the plurality of analytes comprises a nucleic acid. In some embodiments, the nucleic acid comprises a DNA or RNA. In some embodiments, the first tailed primer and said second tailed primer each further comprise a third region 5′ to said second and first region. In some embodiments, c) comprises annealing said plurality of second primers to said third region. In some embodiments, c) comprises annealing second primers of the plurality of second primers to at least a portion of said second region of said first tailed primer or said second tailed primer. In some embodiments, the first set of probe binding sites and said the second set of probe binding sites comprise a same sequence. In some embodiments, the first set of probe binding sites and said the second set of probe binding sites comprise a same probe binding sequence. In some embodiments, the one or more analytes comprises two or more analytes. In some embodiments, the two or more analytes comprises a first analyte present at a concentration of no more than 5% of a concentration of a second analyte. In some embodiments, the two or more analytes comprises a first analyte present at a concentration of no more than 2% of a concentration of a second analyte. In some embodiments, the two or more analytes comprises a first analyte present at a concentration of no more than 1% of a concentration of a second analyte. In some embodiments, the two or more analytes comprises a first analyte present at a concentration of no more than 0.1% of a concentration of a second analyte. In some embodiments, the one or more analytes are detected at a sensitivity of a least 95%. In some embodiments, the one or more analytes are detected at a sensitivity of a least 99%.

In another aspect, the present disclosure provide an oligonucleotide primer comprising a first region substantially complementary to a nucleic acid target and a second region comprising two or more probe binding sites, wherein one or more of the two or more probe binding site is configured to anneal to an oligonucleotide probe that generates a detectable signal upon either of: (i) binding to said probe binding sites or (ii) degradation of said oligonucleotide probe. In some embodiments, the oligonucleotide primer further comprises a third region 5′ to said first region and said second region. In some embodiments, the first region is 3′ to said second region. In some embodiments, the two or more probe binding sites comprises three or more probe binding sites. In some embodiments, the two or more probe binding sites comprises four probe binding sites.

In another aspect, the present disclosure provides a composition comprising: (i) an oligonucleotide comprising a first region substantially complementary to a first region of a nucleic acid target and a second region comprising two or more probe binding sites; and (ii) a plurality of probes, wherein a probe of said plurality of probes is configured to anneal to said one or more probe binding sites, or complements thereof.

In some embodiments, the oligonucleotide further comprises a universal primer binding region 5′ to said first region and said second region. In some embodiments, the composition further comprises a universal primer oligonucleotide comprising a region substantially complementary or homologous to said universal primer binding. In some embodiments, the composition further comprises a primer binding oligonucleotide comprising a region substantially complementary or homologous to a sequence of said oligonucleotide. In some embodiments, the first region is 3′ to said second region. In some embodiments, the oligonucleotide further comprises a third region 5′ to said first region and said second region, wherein said third region is substantially complementary to said nucleic acid target at a sequence different than said first region of said nucleic acid target. In some embodiments, the two or more probe binding sites comprises three or more probe binding sites. In some embodiments, the two or more probe binding sites comprises four probe binding sites. In some embodiments, a first probe of the plurality of probes is present in a same concentration as a concentration of a second probe of the plurality of probes. In some embodiments, a first probe of the plurality of probes is present in a different concentration as a concentration of a second probe of the plurality of probes. In some embodiments, the concentration of a first probe of the plurality of probes is greater the two times the concentration of a second probe of the plurality of probes. In some embodiments, the method further comprises a reverse oligonucleotide comprising a region substantially complementary to a second region of said nucleic acid target. In some embodiments, the composition further comprises a second oligonucleotide comprising a first region substantially complementary to a first region of a second nucleic acid target and a second region comprising one or more probe binding sites. In some embodiments, the composition further comprises a tail oligonucleotide comprising a first region substantially complementary to a first region of a second nucleic acid target and a second region comprising one or more probe binding sites.

In another aspect, the present disclosure provides a composition comprising: (i) a plurality of oligonucleotides, wherein each oligonucleotide of said plurality of oligonucleotides comprises a first region substantially complementary to a nucleic acid target and a second region comprising a set of one or more probe binding sites, wherein the set of one or more probe binding sites for a first oligonucleotide is different than the set of one or more probe binding sites for a second oligonucleotide, wherein said set of one or more probe binding sites for said first oligonucleotide and said set of one or more probe binding sites for a second oligonucleotide comprise a probe binding site that is the same; and (ii) a plurality of probes, wherein a probe of said plurality of probes is configured to anneal to said one or more probe binding sites, or complements thereof. In some embodiments, each oligonucleotide further comprises a universal primer binding 5′ to said first region and said second region. In some embodiments, the composition further comprises a universal primer oligonucleotide comprising a region substantially complementary or homologous to said universal primer binding region. In some embodiments, the composition further comprises a primer binding oligonucleotide comprising a region substantially complementary or homologous to a sequence of said plurality of oligonucleotides. In some embodiments, the first region is 3′ to said second region. In some embodiments, the oligonucleotide further comprises a third region 5′ to said first region and said second region, wherein said third region is substantially complementary to said nucleic acid target at a sequence different than said first region of said nucleic acid target. In some embodiments, the one or more probe binding sites comprises two or more probe binding sites. In some embodiments, the one or more probe binding sites comprises three or more probe binding sites. In some embodiments, the one or more probe binding sites comprises four probe binding sites. In some embodiments, a first probe of the plurality of probes is present in a same concentration as a concentration of a second probe of the plurality of probes. In some embodiments, a first probe of the plurality of probes is present in a different concentration as a concentration of a second probe of the plurality of probes. In some embodiments, the concentration of a first probe of the plurality of probes is greater the two times the concentration of a second probe of the plurality of probes. In some embodiments, the composition further comprises a reverse oligonucleotide comprising a region substantially complementary to a second region of said nucleic acid target. In some embodiments, the composition further comprises a secondary oligonucleotide comprising a first region substantially complementary to a first region of a second nucleic acid target and a second region comprising one or more probe binding sites.

In an aspect, the present disclosure provides a composition comprising: (i) a plurality of probe oligonucleotides, wherein at least two probe oligonucleotides of said plurality of probe oligonucleotides comprise a same nucleic acid sequence and a fluorophore and wherein each fluorophore of said at least two probe oligonucleotide is different. In some embodiments, the composition further comprises a first primer configured to anneal to a first region of an analyte, and a second primer configured to anneal to a second region of an analyte. In some embodiments, the composition further comprises a third primer configured to anneal to a first region of a second analyte, and a fourth primer configured to anneal to a second region of second analyte. In some embodiments, at least three probe oligonucleotides of said plurality of probes comprise a same nucleic acid sequence and a fluorophore and wherein each fluorophore of said at least three probe oligonucleotide is different. In some embodiments, at least four probe oligonucleotides of said plurality of probes comprise a same nucleic acid sequence and a fluorophore and wherein each fluorophore of said at least four probe oligonucleotide is different. In some embodiments, each probe of said plurality of probe oligonucleotides is at a same concentration as other probes comprising different sequences.

In an aspect, the present disclosure provides a method of identifying the presence of an analyte in a sample, the method comprising: a) providing to said sample an oligonucleotide comprising, from 5′ to 3′, a first region that hybridizes to said analyte at a first sequence, a second region comprising two or more distinct probe binding sites, and a third region that hybridizes to said analyte at a second sequence adjacent to said first sequence; b) subjecting said oligonucleotide to a circularization reaction, thereby generating a circularized probe binding nucleic acid; d) annealing a (i) primer oligonucleotide and (ii) one or more probes to said circularized probe binding nucleic acid, or derivatives thereof; e) subjecting said primer oligonucleotide to an extension reaction, thereby generating one or more signals; and f) identifying the presence of said analyte based at least on detection of an intensity level and wavelength of said one or more signals. In some embodiments, prior to (b), said oligonucleotide hybridizes to said analyte at said first sequence and said second sequence. In some embodiments, the method further comprises, subsequent to (b) and prior to (d), cleaving said circularized probe binding nucleic acids to generate a linearized probe binding nucleic acid, wherein (d) comprises annealing said primer oligonucleotide and said one or more probes to said linearized probe binding nucleic acids. In some embodiments, the method further comprises subsequent to (b), and prior to (d), subjecting said sample to an exonuclease reaction. In some embodiments, the exonuclease reaction degrades oligonucleotides that fail to anneal to an analyte. In some embodiments, the one or more signals is detected in more than one channel. In some embodiments, the one or more signals is two or more signals. In some embodiments, the one or more signals is three or more signals. In some embodiments, the said one or more signals is four or more signals. In some embodiments, the one or more signals is detected in more than two channels. In some embodiments, the one or more signals is detected in more than three channels. In some embodiments, a signal of said one or more signals is detected at an intensity level of 1i. In some embodiments, a signal of said one or more signals is detected at an intensity level of 2i. In some embodiments, the one or more signals is generated from more than one probe that binds to said probe binding nucleic acid. In some embodiments, the one or more signals is generated via degradation of one or more probes that binds to said probe binding nucleic acid. In some embodiments, a probe of said one or more probes comprises fluorophore. In some embodiments, the probe comprises a quencher. In some embodiments, the fluorophore is selected from a group consisting of FAM, HEX, TAMRA, Cy5, and combinations thereof. In some embodiments, the second region comprises three or more distinct probe binding sites. In some embodiments, the second region comprises four distinct probe binding sites. In some embodiments, prior to d), providing a first probe that binds to first probe binding site of said probe binding nucleic acid and a second probe that binds to a second probe binding site of said probe binding nucleic acid. In some embodiments, the first probe is present in a same concentration as a concentration of said second probe. In some embodiments, the first probe is present in a different concentration as a concentration of said second probe. In some embodiments, the first probe is present in a greater concentration than a concentration of said second probe. In some embodiments, the first probe is present in more than two times greater concentration than a concentration of said second probes. In some embodiments, e) further comprises providing a reverse primer that hybridizes to said linearized probe binding nucleic acid. In some embodiments, e) comprises performing an amplification reaction to generate a plurality of amplicons of said linearized probe binding nucleic acid. In some embodiments, the method further comprises, subsequent to a) and prior to b) generating a plurality of partitions. In some embodiments, the analyte comprises a nucleic acid. In some embodiments, the nucleic acid comprises a DNA or RNA. In some embodiments, the first primer oligonucleotide further comprises a third region 5′ to said second and first region. In some embodiments, d) comprises annealing said second primer oligonucleotide to said third region. In some embodiments, d) comprises annealing said second primer oligonucleotide to at least a portion of said second region. In some embodiments, the method further comprises, identifying the presence of a second analyte, wherein a) comprises providing a third primer oligonucleotide comprising a first region that hybridizes to said second analyte and a second region comprising one or more probe binding sites. In some embodiments, the third primer oligonucleotide comprises from 5′ to 3′, a first region that hybridizes to said second analyte at a first sequence, a second region comprising one or more probe binding sites, and a third region configured to hybridize to said second analyte at a second sequence adjacent to said first sequence of said second analyte. In some embodiments, the analyte is detected at a sensitivity of a least 95%. In some embodiments, the analyte is detected at a sensitivity of a least 99%.

In another aspect, the present disclosure provides a method of identifying the presence of one or more analytes of a plurality of analytes in a sample, the method comprising: a) providing to said sample a plurality of molecular inversion oligonucleotides wherein a first molecular inversion oligonucleotide of said plurality of molecular inversion oligonucleotides comprises, from 5′ to 3′, a first region that hybridizes a first sequence of said analyte, a second region comprising a first set of one or more probe binding sites, and a third region that hybridizes to a second sequence of said analyte that is adjacent to said first sequence of said analyte, and a second molecular inversion oligonucleotide of said plurality of molecular inversion oligonucleotides comprises, from 5′ to 3′, a first region that hybridizes a first sequence of a second analyte, a second region comprising a second set of one or more probe binding sites, and a third region that hybridizes to a second sequence of said second analyte that is adjacent to said first sequence of said second analyte, wherein said first set of probe binding sites and said second set of probe binding sites are not identical; b) subjecting said plurality of molecular inversion oligonucleotides to circularization reactions, thereby generating circularized probe binding nucleic acids; c) cleaving said circularized probe binding nucleic acids to generate linearized probe binding nucleic acids; d) annealing a plurality of second primers to said linearized probe binding nucleic acids; e) subjecting said plurality of second primers to extension reactions to generate (i) if the first analyte is present, a first set of one or more signals, and (ii) if the second analyte is present, a second set of one or more signals; and f) based at least on if present, said first set of one or more signals and, if present, said second set of one or more signals, identifying the presence of one or more analytes of the plurality of analytes. In some embodiments, e) comprises generating (i) if said first analyte is present, a first set of one or more signals from one or more probes that bind said first set of probe binding sites, and (ii) if said second analyte is present, a second set of one or more signals from one or more probes that bind said second set of probe binding sites. In some embodiments, the first set of one or more signals or second set of one or more signals is detected in more than one channel. In some embodiments, the first set of one or more signals or second set of one or more signals is detected in more than two channels. In some embodiments, the first set of one or more signals or second set of one or more signals is detected in more than three channels. In some embodiments, the first set of one or more signals or second set of one or more signals is detected in more than four channels. In some embodiments, the identifying the presence of the plurality of analytes is based at least on identifying an intensity level of the signals of the first or second set of one or more signals. In some embodiments, a signal of said first or second set of one or more signals is detected at an intensity level of 1i. In some embodiments, a signal of said first or second set of one or more signals is detected at an intensity level of 2i. In some embodiments, a first signal of the first set of one or more signals is detected in a first channel at an intensity of 1i and a second signal of the first set of one or more signals is detecting in a second channel at an intensity of 1i. In some embodiments, a first signal of the first set of one or more signals is detected in a first channel at an intensity of 1i and a second signal of the first set of one or more signals is detecting in a second channel at an intensity of 2i. In some embodiments, the first or second set of one or more signals is generated from more than one probe that binds to said probe binding nucleic acid. In some embodiments, the first or second set of one or more signals is generated via degradation of said a probe that binds to said probe binding nucleic acid. In some embodiments, the method further comprises, subsequent to c) and prior to e), annealing one or more probes to the first or second set of probe binding sites. In some embodiments, a probe of said one or more probes comprises fluorophore. In some embodiments, the probe comprises a quencher. In some embodiments, the fluorophore is selected from a group consisting of FAM, HEX, TAMRA, Cy5, and combinations thereof. In some embodiments, the second region of the first molecular inversion oligonucleotide or the second molecular inversion oligonucleotide comprises two or more probe binding sites. In some embodiments, the second region of the first molecular inversion oligonucleotide or the second molecular inversion oligonucleotide comprises three or more probe binding sites. In some embodiments, the second region of the first molecular inversion oligonucleotide or the second molecular inversion oligonucleotide comprises four probe binding sites. In some embodiments, the method comprises, prior to d), providing a first probe that binds to first probe binding site of said probe binding nucleic acids and a second probe that binds to a second probe binding site of said probe binding nucleic acids. In some embodiments, the first probe is present in a same concentration as a concentration of said second probe. In some embodiments, the first probe is present in a different concentration as a concentration of said second probe. In some embodiments, the first probe is present in a greater concentration than a concentration of said second probe. In some embodiments, the first probe is present in more than 2 times greater concentration than a concentration of said second probes. In some embodiments, d) further comprises providing a third primer oligonucleotide that hybridizes to said linearized probe binding nucleic acids. In some embodiments, the e) comprises performing an amplification reaction to generate a plurality of amplicons of said linearized probe binding nucleic acids. In some embodiments, the method further comprises, subsequent to a) and prior to b) generating a plurality of partitions. In some embodiments, a first partition of said plurality of partition comprises said first analyte and a second partition of said plurality of partition comprises said second analytes. In some embodiments, the first set of one or more signals are generated in said first partition and said second set of signal and generated in said second partition. In some embodiments, the method further comprises, detecting said first set of one or more signals separately from detecting said second set of one or more signals. In some embodiments, the plurality of analytes comprises a nucleic acid. In some embodiments, the nucleic acid comprises a DNA or RNA. In some embodiments, the first molecular inversion oligonucleotide and said second molecular inversion oligonucleotide each further comprise a third region. In some embodiments, c) comprises annealing said plurality of second primers to said third region. In some embodiments, c) comprises annealing second primers of the plurality of second primers to at least a portion of said second region of said first molecular inversion oligonucleotide or said second molecular inversion oligonucleotide. In some embodiments, the first set of probe binding sites and said the second set of probe binding sites comprise a same sequence. In some embodiments, the first set of probe binding sites and said the second set of probe binding sites comprise a same probe binding sequence. In some embodiments, the one or more analytes comprises two or more analytes. In some embodiments, the two or more analytes comprises a first analyte present at a concentration of no more than 5% of a concentration of a second analyte. In some embodiments, the two or more analytes comprises a first analyte present at a concentration of no more than 2% of a concentration of a second analyte. In some embodiments, the two or more analytes comprises a first analyte present at a concentration of no more than 1% of a concentration of a second analyte. In some embodiments, the two or more analytes comprises a first analyte present at a concentration of no more than 0.1% of a concentration of a second analyte. In some embodiments, the one or more analytes are detected at a sensitivity of a least 95%. In some embodiments, the one or more analytes are detected at a sensitivity of a least 99%.

In another aspect, the present disclosure provides an oligonucleotide comprising, from 5′ to 3′, a first region configured to hybridize to said analyte at a first sequence, a second region comprising two or more distinct probe binding sites, and a third region configured to hybridize to said analyte at a second sequence adjacent to said first sequence, wherein one or more of the two or more probe binding site is configured to anneal to an oligonucleotide probe that generates a detectable signal upon either of: (i) binding to said probe binding sites or (ii) degradation of said oligonucleotide probe. In some embodiments, the two or more probe binding sites comprises three or more probe binding sites. In some embodiments, the two or more probe binding sites comprises four probe binding sites.

### BRIEF DESCRIPTION OF THE DRAWINGS

The novel features of the invention are set forth with particularity in the appended claims. “The patent or application file contains at least one drawing executed in color. Copies of this patent or patent application publication with color drawing(s) will be provided by the Office upon request and payment of the necessary fee. A better understanding of the features and advantages of the present invention will be obtained by reference to the following detailed description that sets forth illustrative embodiments, in which the principles of the invention are utilized, and the accompanying drawings (also “figure” and “FIG.” herein), of which:

FIG. 1 shows a schematic of different oligonucleotides probe binding sites.

FIG. 2 shows a schematic of the methods and oligonucleotides of the disclosure.

FIG. 3 shows a schematic of the methods and oligonucleotides of the disclosures including a set of probes.

FIG. 4 shows a schematic of the methods and oligonucleotides of the disclosure including a set of probes.

FIG. 5 shows a schematic of the methods and oligonucleotides of the disclosure including a set of probes.

FIG. 6 shows a schematic of the methods and oligonucleotides of the disclosure including a set of probes.

FIG. 7 shows data demonstrating distinct populations.

FIG. 8A shows a schematic of the methods and oligonucleotides of the disclosure including a set of probes. FIG. 8B shows data demonstrating detection of 3 distinct intensity levels.

FIG. 9 shows data demonstrating detection of different analytes.

FIG. 10 shows a schematic for detection of different analytes.

FIG. 11 shows a schematic of an example oligonucleotide.

FIG. 12 shows an schematic of an example method and example oligonucleotides.

FIG. 13 shows an schematic of an example method and example oligonucleotides.

FIG. 14 shows data relating to detection of multiple different barcodes at various dilutions.

FIG. 15 shows data relating to multiplex detection of different targets in a mixed sample.

FIG. 16 shows an example workflow for detection using oligonucleotides described in this disclosure.

FIG. 17 shows data relating to detection using oligonucleotides of the disclosure.

### DETAILED DESCRIPTION OF THE INVENTION

The following description provides specific details for a comprehensive understanding of, and enabling description for, various embodiments of the technology. It is intended that the terminology used be interpreted in its broadest reasonable manner, even where it is being used in conjunction with a detailed description of certain embodiments.

Before describing the present teachings in detail, it is to be understood that the disclosure is not limited to specific compositions or process steps, and as such, may vary. As used herein, the singular forms “a,” “an,” and “the” are intended to include the plural forms as well, unless the context clearly indicates otherwise. Furthermore, to the extent that the terms “including” “includes,” “having,” “has,” “with,” “such as,” or variants thereof, are used in either the specification and/or the claims, such terms are not limiting and are intended to be inclusive in a manner similar to the term “comprising.” Unless specifically noted, embodiments in the specification that recite “comprising” various components are also contemplated as “consisting of” or “consisting essentially of” the recited components.

Where values are described as ranges, it will be understood that such disclosure includes the disclosure of all possible sub-ranges within such ranges, as well as specific numerical values that fall within such ranges irrespective of whether a specific numerical value or specific sub-range is expressly stated

The term “subject,” as used herein, generally refers to an animal, such as a mammal (e.g, human) or avian (e.g., bird), or other organism, such as a plant. For example, the subject can be a vertebrate, a mammal, a rodent (e.g., a mouse), a primate, a simian or a human. A subject can be a healthy or asymptomatic individual, an individual that has or is suspected of having a disease (e.g., cancer) or a pre-disposition to the disease, and/or an individual that is in need of therapy or suspected of needing therapy. A subject can be a patient.

The term “channel,” “color channel,” or “optical channel”, as used herein, generally refers to a range of wavelengths. The channel may be set or determined based on particular filters which remove or filter out particular wavelengths. The terms “channel,” “color channel,” and “optical channel” can be used interchangeably.

Primers, or “amplification oligomers,” used herein interchangeably, refer to an oligonucleotide or nucleic acid configured to bind to another nucleic acid and facilitate one or more reactions, for example, transcription, nucleic acid synthesis, extension of a nucleic acid primer, and nucleic acid amplification. A primer can be double-stranded. A primer can be single-stranded. A primer can be a forward primer or a reverse primer. A forward primer and a reverse primer can be those which bind to opposite strands of a double-stranded nucleic acid. For example, a forward primer can bind to a region of a first strand (e.g., Watson strand) derived from a nucleic acid, and a reverse primer can bind to a region of a second strand (e.g., Crick strand) derived from the nucleic acid. A forward primer may bind to a region closer to the start site of a gene relative to a reverse primer or may bind closer to the end site of a gene relative to a reverse primer. A forward primer may bind to the coding strand of a nucleic acid or may bind to the non-coding strand of a nucleic acid. A reverse primer may bind to the coding strand of a nucleic acid or may bind to the non-coding strand of a nucleic acid.

Polymerase Chain Reaction (PCR) is a method of exponential amplification of specific nucleic acid target in a reaction mix with a nucleic acid polymerase and primers. Primers are short single stranded oligonucleotides which are complementary to the 3′ sequences of the positive and negative strand of the target sequence. The reaction mix is cycled in repeated heating and cooling steps. The heating cycle denatures or splits a double stranded nucleic acid target into single stranded templates. In the cooling cycle, the primers bind to complementary sequence on the template. After the template is primed the nucleic acid polymerase creates a copy of the original template. Repeated cycling exponentially amplifies the target 2-fold with each cycle leading to approximately a billion-fold increase of the target sequence in 30 cycles (Saiki et al 1988).

Real-Time PCR (qPCR) is a process of monitoring a PCR reaction by recording the fluorescence generated either by an intercalating dye such as SYBR Green or a target-specific reporter probe at each cycle. This is generally performed on a Real-Time PCR instrument that executes thermal cycling of the sample to complete the PCR cycles and at a specified point in each cycle measures the fluorescence of the sample in each channel through a series of excitation/emission filter sets.

Digital PCR (dPCR) is a process of partitioning a sample containing one or more targets into a plurality of partitions (e.g., wells, droplets, etc.), performing a PCR reaction in each partition, and recording the luminescence (e.g., fluorescence) generated by, for example, a target-specific re-porter probe. The use of labeled oligonucleotide probes enables specific detection. dPCR may be used in a variety of nucleic acid detection methods. Digital PCR is generally performed on a digital PCR instrument that measures the fluorescence from each partition in an optical channel through one or more excitation/emission filter sets. Frequently, the target-specific oligonucleotide probe is a short oligonucleotide complementary to one strand of the amplified target. The probe lacks a 3′ hydroxyl and therefore is not extendable by the DNA polymerase. TaqMan (ThermoFisher Scientific) chemistry is a common reporter probe method used for multiplex Real-Time PCR (Holland et al. 1991). The TaqMan oligonucleotide probe is covalently modified with a fluorophore and a quenching tag (i.e., quencher). In this configuration the fluorescence generated by the fluorophore is quenched and is not detected by the real time PCR instrument. When the target of interest is present, the probe oligonucleotide base pairs with the amplified target. While bound, it is digested by the 5′ to 3′ exonuclease activity of the Taq polymerase thereby physically separating the fluorophore from the quencher and liberating signal for detection by the real time PCR instrument.

Provided herein are methods, systems, compositions, and kits for detection and quantification of multiple nucleic acids sequences or analytes in a sample. The methods, systems, compositions, and kits may use oligonucleotides to generate signals for different targets. The oligonucleotides may comprise a target binding region, and one or more probe binding regions. The probe binding regions may be specific to a given target such that for a given target a specific signal is generated. In such a way, multiple targets may be “barcoded” such that a specific signal generated is indicative of the presence of a target.

Traditional TaqMan assays may use a probe that is sequence specific to a given target and may generate a signal based on the presence of binding to a target. The probes may therefore need to be designed to bind to specific sequence and each target may need to be optimized to generate probes that are suitable to a given target. The methods, systems, compositions, and kits may be configured such that universal probes may be used that have a sequence that does not need to be altered depending on the target. This may allow for more flexibility of design and improvements in multiplexed detection of analytes.

Provided herein are tailed primers that comprise a probe binding region comprising one or more probe binding sites. The tailed primers of the disclosure may comprise a target specific region. The tailed primer may anneal to a target and be extended or amplified to generate a nucleic acids comprising nucleic corresponding to the target and nucleic acid corresponding to the probe binding region. The tailed primer may “barcode” the target nucleic acid so that a nucleic acid is produced that can generate a signal indicative of the target. The probe binding regions may bind to universal probes or probes that anneal to sequences in a probe binding regions (e.g., the primer tail) and do not anneal to sequences of the target. Multiple tailed primers may be used to perform multiplexed target detection, wherein the tailed primers have regions that are specific to different targets, and a probe binding region that is different depending on the target. Each target may have a different set of probes or barcode associated with it. Using universal probes may allow for improved multiplex as multiple probe binding region may comprise a same probe binding site, with different regions comprising different combinations of probe binding sites.

Provided herein are molecular inversion probe-like oligonucleotides that comprise a probe binding region comprising one or more probe binding sites. The molecular inversion probe-like oligonucleotides of the disclosure may comprise a first target specific region, a probe binding region comprising one or more probe binding site, and a second target specific region. The molecular inversion probe-like oligonucleotides may “barcode” the target nucleic acid so that a nucleic acid is produced that can generate a signal indicative of the target. The probe binding regions may bind to universal probes or probes that anneal to sequences in a probe binding regions (e.g., in the oligonucleotides) and do not anneal to sequences of the target. Multiple molecular inversion probe-like oligonucleotides may be used to perform multiplexed target detection, wherein the molecular inversion probe-like oligonucleotides have regions that are specific to different targets, and a probe binding region that is different depending on the target. Each target may have a different set of probes or barcode associated with it. Using universal probes may allow for improved multiplex as multiple probe binding region may comprise a same probe binding site, with different regions comprising different combinations of probe binding sites.

In some examples, each analyte to be detected is encoded as a value of a signal (e.g., intensity), where the values are assigned so that the results of the assay unambiguously indicate the presence or absence of the analytes being assayed. In other examples, each analyte to be detected is encoded as values in each of at least two components of a signal (e.g., intensity and wavelength). The at least two components of a signal may be orthogonal. The signal may comprise a particular intensity level. An intensity level may be at an intensity of 1i, wherein “i” represents an expected or fixed intensity. An intensity level may be at an intensity level of 2i, such that the intensity is twice that of an intensity level of “i”. In this manner, intensity levels may be used to encode for or otherwise be associated with a given analyte or set of analytes. For example, a first analyte may be represented by or associated with a signal intensity of i and a second analyte may be represented by or associated with a signal intensity of 2i.

A target analyte may be represented, associated, or encoded as a set of signals of wavelengths and intensities. For example, a target analyte may be encoded using 4 different wavelengths (or wavelength ranges) and intensities. For example, with four universal reporter sites in the backbone (chosen from a set of 8 universal reporter sequences), 3 amplitude levels (0, 1i, 2i), and four color channels, up to 80 unique targets can be interrogated with a standard ternary encoding scheme.

Table 1 shows an example of different encoding or association for targets using 4 different channels and intensities of 0, 1 or 2.

| Target # | Channel 1 | Channel 2 | Channel 3 | Channel 4 | |
|---|---|---|---|---|---|
| 1 | 1 | 0 | 0 | 0 | all 1i |
| 2 | 0 | 1 | 0 | 0 | digital |
| 3 | 0 | 0 | 1 | 0 | encoding |
| 4 | 0 | 0 | 0 | 1 | |
| 5 | 1 | 1 | 0 | 0 | |
| 6 | 1 | 0 | 1 | 0 | |
| 7 | 1 | 0 | 0 | 1 | |
| 8 | 0 | 1 | 1 | 0 | |
| 9 | 0 | 0 | 1 | 1 | |
| 10 | 0 | 1 | 0 | 1 | |
| 11 | 1 | 1 | 1 | 0 | |
| 12 | 0 | 1 | 1 | 1 | |
| 13 | 1 | 0 | 1 | 1 | |
| 14 | 1 | 1 | 0 | 1 | |
| 15 | 1 | 1 | 1 | 1 | |
| 16 | 2 | 0 | 0 | 0 | all 2i |
| 17 | 0 | 2 | 0 | 0 | digital |
| 18 | 0 | 0 | 2 | 0 | encoding |
| 19 | 0 | 0 | 0 | 2 | |
| 20 | 2 | 2 | 0 | 0 | |
| 21 | 2 | 0 | 2 | 0 | |
| 22 | 2 | 0 | 0 | 2 | |
| 23 | 0 | 2 | 2 | 0 | |
| 24 | 0 | 0 | 2 | 2 | |
| 25 | 0 | 2 | 0 | 2 | |
| 26 | 2 | 2 | 2 | 0 | |
| 27 | 0 | 2 | 2 | 2 | |
| 28 | 2 | 0 | 2 | 2 | |
| 29 | 2 | 2 | 0 | 2 | |
| 30 | 2 | 2 | 2 | 2 | |
| 31 | 1 | 2 | 0 | 0 | all li 2i |
| 32 | 2 | 1 | 0 | 0 | pairwise |
| 33 | 1 | 0 | 2 | 0 | digital |
| 34 | 2 | 0 | 1 | 0 | encoding |
| 35 | 1 | 0 | 0 | 2 | |
| 36 | 2 | 0 | 0 | 1 | |
| 37 | 0 | 1 | 2 | 0 | |
| 38 | 0 | 2 | 1 | 0 | |
| 39 | 0 | 1 | 0 | 2 | |
| 40 | 0 | 2 | 0 | 1 | |
| 41 | 0 | 0 | 1 | 2 | |
| 42 | 0 | 0 | 2 | 1 | |
| 43 | 1 | 1 | 2 | 0 | all li 2i |
| 44 | 1 | 1 | 1 | 0 | triple |
| 45 | 2 | 1 | 1 | 0 | encoding |
| 46 | 1 | 2 | 2 | 0 | |
| 47 | 2 | 1 | 2 | 0 | |
| 48 | 0 | 1 | 1 | 2 | |
| 49 | 0 | 1 | 2 | 2 | |
| 50 | 0 | 1 | 2 | 1 | |
| 51 | 0 | 2 | 1 | 1 | |
| 52 | 0 | 1 | 2 | 2 | |
| 53 | 0 | 2 | 1 | 2 | |
| 54 | 0 | 2 | 2 | 1 | |
| 55 | 1 | 0 | 2 | 1 | |
| 56 | 1 | 0 | 1 | 2 | |
| 57 | 2 | 0 | 1 | 1 | |
| 58 | 2 | 0 | 2 | 1 | |
| 59 | 2 | 0 | 1 | 2 | |
| 60 | 1 | 0 | 2 | 2 | |
| 61 | 1 | 1 | 0 | 2 | |
| 62 | 1 | 2 | 0 | 1 | |
| 63 | 2 | 1 | 0 | 1 | |
| 64 | 1 | 2 | 0 | 2 | |
| 65 | 2 | 1 | 0 | 2 | |
| 66 | 2 | 2 | 0 | 1 | |
| 67 | 1 | 1 | 1 | 2 | all li 2i |
| 68 | 1 | 1 | 2 | 1 | quad |
| 69 | 1 | 1 | 2 | 2 | encoding |
| 70 | 1 | 2 | 1 | 1 | |
| 71 | 1 | 2 | 1 | 2 | |
| 72 | 1 | 2 | 2 | 1 | |
| 73 | 1 | 2 | 2 | 2 | |
| 74 | 2 | 1 | 1 | 1 | |
| 75 | 2 | 1 | 1 | 2 | |
| 76 | 2 | 1 | 2 | 1 | |
| 77 | 2 | 1 | 2 | 2 | |
| 78 | 2 | 2 | 1 | 1 | |
| 79 | 2 | 2 | 1 | 2 | |
| 80 | 2 | 2 | 2 | 1 | |

With a quaternary encoding scheme (4 levels per color channel) up to 255 unique targets could be encoded with four universal reporter sites in the backbone (selected from a set of 12 sequences). The methods can scale with the addition of more color channels and amplitudes levels. Additionally, the analysis of the signals is not limited to a digital PCR format and may be extended to methods that can identify the presence or absence of a multiple nucleic acid sequence in a single template molecule may be used, for example, nanopore sequencing, single molecule sequencing single molecule FISH, or loop mediated isothermal amplification.

After encoding of the analytes, a sample may be provided wherein the sample comprises or may comprise at least one of the encoded analytes. The sample may be contacted with an analyte-specific reagent or reagents that generate a particular signal, as specified for each analyte in the coding scheme, in the presence of an analyte. A reagent may be any suitable reagent that is capable of generating such a signal in the presence of its corresponding analyte, for example, an oligonucleotide probe attached to a fluorophore and a quencher (e.g., a TAQMAN probe). If the reagent is an oligonucleotide probe attached to a fluorophore and a quencher, a nucleic acid amplification may be performed to generate the signal.

After addition of the reagent(s), the signal may be quantified. In some cases, this quantification is performed by measuring one component of the signal (e.g., fluorescence intensity) and determining the presence and absence of certain analytes based on the values used to encode the presence of each analyte and the cumulative value of the signal.

In some cases, at least two components of a signal (e.g., intensity and wavelength) are cumulatively measured for the sample. This measurement can be performed, for example, by measuring the intensity at a particular wavelength or the intensity within a particular range of wavelengths. The presence or absence of an analyte may then be determined based on the values of each of the at least two components of the signal and the values used to encode the presence of the analyte. In some cases, a component of a signal (e.g., intensity) is measured for the sample or for a partition. The signal intensity may be used to encode or otherwise indicate the presence of an analyte or unique combination of analytes.

The methods, system, compositions, and kits, may be used in conjunction with digital PCR assays or other assays with a single molecule counting-based readout mechanism; e.g. digital PCR, digital droplet PCR, single molecule DNA sequencing, or mass spectrometry. As each analyte can be separately analyzed and each analyte can have a separate signal readout, multiplex detection can be performed. For example, first partition can have an analyte that generates a signal (e.g., set of wavelengths and intensities) indicative of the first analyte and a second partition can have an analyte that generates a signal indicative of the second analyte. Analysis of the two partitions can allow for detection of both analytes.

In various aspects disclosed herein, the results of the assay may be used to determine that a nucleic acid sequence or infectious pathogen is present in a subject or sample.

Methods as described herein may be used to identify or quantify 1, 2, 3, 4, 5, 6, 7, 8, 9, 10, or more analytes in a sample volume. First, a mixture may be provided comprising a plurality of nucleic acid molecules and a plurality of oligonucleotide probes. The plurality of nucleic acid molecules may be derived from, and/or may correspond with, the nucleic acid target in the sample. The plurality of oligonucleotide probes may each correspond to a different region of the nucleic acid target. The oligonucleotide probes be able to hybridize to more than one nucleic acid target. The mixture may further comprise other reagents (e.g., amplification reagents) including, for example, oligonucleotide primers, dNTPs, a nucleic acid enzyme (e.g., a polymerase), and salts (e.g., Ca2+, Mg2+, etc.). A oligonucleotide primer of the oligonucleotide primers may be able to hybridize to more than one nucleic acid target. The mixture may be partitioned into a plurality of partitions. Next, the mixture may be used in a quantitative Polymerase Chain Reaction (qPCR), whereby a plurality of signals may be generated. The plurality of signals may be detectable in multiple color channels. Based on the detecting, the nucleic acid target in the sample may be quantified. A signal of the plurality of the signal may be detectable in only one color channel. For example, a first signal of the plurality of signals is detected in multiple color channels, and a second signal is detectable in only one color channel, and the analytes correlated to the first and second signals may be quantified. In another example, a first signal of the plurality of the signals is detected in a first two color channels and a second signal of the plurality of signals is detected in a second two color channels, and at least one of the channels in the first two color channels and the second two color channels is the same or substantially the same color channel. In another example, a first signal may be generated that corresponds to a first analyte and a second signal may be generated that corresponds to a second analyte. The different signals may then be analyzed to determine the presence of the analytes or quantify the analytes, as described elsewhere herein. In the case of a partitioned sample, the plurality of signals may be detected in multiple partitions of the plurality of partitions. A signal may be detected or measured at least 1, 2, 3, 4, 5, 6, 7, 8, 9, 10, 11, 12, 13, 14, 15, 20, 30, 40, 50, or more channels. A signal may be detected or measured in no more than 1, 2, 3, 4, 5, 6, 7, 8, 9, 10, 11, 12, 13, 14, 15, 20, 30, 40, 50, or less channels.

In one example of dPCR, a single sample containing at least one nucleic acid target sequence, at least one amplification oligomer, at least one detection oligonucleotide, dNTPs, a thermostable DNA polymerase, and other PCR reagents may be partitioned into approximately 20,000 evenly sized partitions. Generally, each partition may receive a single template of the nucleic acid target sequence. However, statistically, some partitions may receive more than one copy of a nucleic acid target template, while other partitions may not receive any target template. Some partitions may receive 1, 2, 3, 4, 5, 6, 7, 8, 9, 10 or more nucleic acid targets.

The plurality of signals may be generated by one or more of the plurality of probes from the mixture. The plurality of signals may be generated by nucleic acid amplification (e.g., PCR) of the plurality of nucleic acid molecules. Nucleic acid amplification may degrade the plurality of oligonucleotide probes (e.g., by activity of a nucleic acid enzyme), thereby generating the plurality of signals. A plurality of signals may be a plurality of fluorescent signals, a plurality of chemiluminescent signals, or a combination thereof.

In multiple aspects as described herein, signals and data relating to the detection of the signals are subjected to processing in order for the signals and data to be used for subsequent steps or downstream methods. The processing may use mathematical algorithms to analyze or process the signal data. In some case, the processing may use data obtained from the instrument or detector. The processing may use data obtained from multiple channels, or a single channel. In some cases, the processing may use data from channels that are not expected to correlate with a signal from a given probe or fluorophore. For example, the data may include data obtained from a reference channel in which a background signal is obtained. The processing may use data obtained from all available channels of a given detection device.

In some cases, the sample further comprises an additional plurality of nucleic acid molecules and an additional plurality of oligonucleotide probes. The additional plurality of nucleic acid molecules may be derived from and/or correspond with an additional nucleic acid target. The additional plurality of oligonucleotide probes may each correspond to a different region of the additional nucleic acid target.

In various aspects, nucleic acid molecules may be quantified. The quantification may be an absolute quantification. For example, the molarity of a starting amount of a nucleic acid may be determined. This may be determined using a reference condition or amount with a known molarity of nucleic acid. The quantification may be a relative quantification. For example, a second nucleic acid may be determined to have a larger starting amount than a first nucleic acid.

A sample may be a biological sample. A sample may be derived from a biological sample. A biological sample may be, for example, blood, plasma, serum, urine, saliva, mucosal excretions, sputum, stool or tears. A biological sample may be a fluid sample. A fluid sample may be blood or plasma. A biological sample may comprise cell-free nucleic acid (e.g., cell-free RNA, cell-free DNA, etc.). A nucleic acid target may be a nucleic acid from a pathogen (e.g., virus, bacteria, etc.). A nucleic acid target may be a nucleic acid suspected of comprising one or more mutations.

In various aspects described herein a sample is a collected from a subject or a plurality of subjects. The biological sample may be collected using a sample collection tube or vessel. The biological sample may be collected using a sample collection tool. The sample collection tool may comprise swab. The sample may be collected by the subject without the help of another individual. The sample may be collected in a subject's home away from a medical facility. The sample may be collected based on a set of instructions provided in a kit to the subject.

In various aspects described herein, the results of the assay may be outputted as a report. The report may be outputted to a remote computer database. The report may be outputted such that it is accessible to the subject. The report may be outputted such that it is accessible to a medical provider or to an institution. The report may be accessible via a smart phone. The report may be accessible via an application. The report may be used in the conjunction with a subject medical record. The report may comprise medical recommendations.

In various aspects, partitioning is performed and partitions are generated. Reactions described elsewhere in the specification may be performed in the partitions. For example, a sample containing at least one nucleic acid target sequence, at least one amplification oligonucleotide, at least one detection oligonucleotide, dNTPs, a thermostable DNA polymerase, and other PCR reagents may be partitioned into a plurality of partitions. Generally, each partition may receive a single template of the nucleic acid target sequence. However, statistically, some partitions may receive more than one copy of a nucleic acid target template, while other partitions may not receive any target template. The detection oligonucleotide or amplification oligonucleotide may be configured to bind to multiple analytes. The partitions may each comprise at most one analyte. In each partition, reaction may be performed to generate a signal and may indicate the analyte in a given partition.

In some embodiments, the present disclosure provides a multiplexed assay for simultaneous amplification, detection, and or/quantification of one or more analyte in a sample. The one or more analytes may comprise different sequences from one another.

In some cases, assays may be run using the reagents in the chemical composition. Assay may use a reagent to perform a reaction. The reaction may comprise a hybridization reaction. For example, the reagent may comprise a nucleic acid and hybridize with another nucleic acid. The reaction may comprise an extension reaction. For example, the reaction may comprise extending a nucleic molecule by the addition of a nucleotide. The reaction may comprise a polymerase chain reaction.

Methods as described herein may be performed without the use of immobilization, separation, mass spectrometry, or melting curve analysis. For example, the sample reagents and analytes may all be in solution. The analytes may be analyzed without needing to purify or physically separate the analytes from one another. Identification of the analytes may be performed without obtaining a mass of the analytes via mass spectrometry or any similar technique. Additionally, the methods may be used without observing a melting reaction and plotting the signal against a temperature. For example, an analyte may be identified without subjecting the analyte to temperature gradient in order to analyze a specific temperature in which an analyte goes through a physical or chemical change. The methods as described herein may be corroborated via techniques using immobilization, separation, mass spectrometry, or melting curve analysis. For example, the melting curve may be used to verify a number of different amplicons or detecting a presence of an amplicon.

Any number of nucleic acid targets may be detected or quantified using assays of the present disclosure. In some cases, an assay may unambiguously detect at least 3, 4, 5, 6, 7, 8, 9, 10, 11, 12, 13, 14, 15, 20, 30, 40, 50, 60, 70, or 80 nucleic acid targets, or more. In some cases, an assay may unambiguously detect at most 50, 40, 30, 20, 15, 14, 13, 12, 11, 10, 9, 8, 7, 6, 5, 4, or 3 nucleic acid targets. An assay may comprise any number of reactions, where the results of the reactions together identify a plurality of nucleic acid targets, in any combination of presence or absence. An assay may comprise 2, 3, 4, 5, 6, 7, 8, 9, 10 reactions, or more. Each reaction may be individually incapable of non-degenerately detecting the presence or absence of any combination of nucleic acid targets. However, the results of each reaction together may unambiguously detect the presence or absence of each of the nucleic acid targets.

Reactions may be performed in the same sample solution volume. For example, a first reaction may generate a fluorescent signal in a at least a first color channel, while a second reaction may generate a fluorescent signal in a second color channel, thereby generating two measurements for comparison. Alternatively, reactions may be performed in different sample solution volumes. For example, a first reaction may be performed in a first sample solution volume and generate a fluorescent signal in at least two channels, and a second reaction may be performed in a second sample solution volume and generate a fluorescent signal in the same color channel or a different color channel, thereby generating two measurements for comparison.

A reaction may be performed in a single partition (e.g., a well). A reaction may be performed in a plurality of partitions (e.g., a plurality of droplets). A reaction may comprise polymerase chain reaction (PCR). A reaction may comprise, for example, quantitative PCR (qPCR), digital PCR (dPCR), or droplet digital PCR (ddPCR). In some cases, a single reaction comprises a single PCR reaction (e.g., qPCR, dPCR, ddPCR). The results of two or more reactions (e.g., PCR reactions) may be compared, thereby unambiguously detecting the presence or absence of any combination of a plurality of analytes.

In some aspects, the present disclosure provides methods for detecting the presence or absence of a plurality of analytes in a sample. Analytes may be polynucleotide analytes (e.g, DNA, RNA, etc.). First, a subset of a sample may be contacted with a first plurality of hybridization probes. Each hybridization probe may correspond to a polynucleotide analyte (e.g, may bind to a region of a polynucleotide analyte). A first cumulative signal measurement may be generated comprising one or more signals generated from the first plurality of hybridization probes, The first cumulative signal measurement may fail to non-degenerately identify the presence or absence of any combination of the at least three polynucleotide analytes. Next, one or more additional subsets of the sample may be contacted with one or more additional pluralities of hybridization probes. Each hybridization probe may correspond to a polynucleotide analyte (e.g, may bind to a region of a polynucleotide analyte). One or more additional cumulative signal measurements may be generated, each comprising one or more additional signals generated from the one or more additional pluralities of hybridization probes. The one or more additional cumulative signal measurements may fail to non-degenerately identify the presence or absence of any combination of the at least three polynucleotide analytes. Finally, the first cumulative signal measurement to the one or more additional cumulative signal measurements may be compared. The comparison may uniquely identify any combination of polynucleotide analytes in the sample. The methods may be performed without performing one or more additional reactions to resolve the ambiguity. The quantitation may be performed without unambiguously identifying the combination of a presence or absence in a given sample or partition.

In some aspects, the present disclosure provides methods for detecting the presence or absence of a plurality of analytes or quantifying the plurality of analytes in a sample solution volume. Analytes may be polynucleotide analytes (e.g., DNA, RNA, etc.). First, a sample solution volume comprising, or potentially comprising, a plurality of analytes may be provided. The sample solution may be derived from a sample, for example, a sample from a subject. Next, the sample solution volume may be contacted with a plurality of hybridization probes, which may be excited to generate a cumulative signal measurement if one or more of the plurality of analytes is present in the sample solution volume. The cumulative signal measurement may comprise an ambiguity. The ambiguity may be a signal intensity level corresponding to more than one combination of analytes. Next, a set of information regarding the polynucleotide analytes may be received. The set of information may comprise, for example, an additional cumulative signal measurement, a statistical table, or a desired clinical outcome. The cumulative signal measurement may be compared to the set of information, where the results of the comparing resolve the ambiguity, thereby detecting the presence or absence of analytes. Quantification may be performed without using results to resolve the ambiguity.

An oligonucleotide probe may be labeled with a detectable label. A detectable label may comprise a fluorophore. Fluorescent molecules may be excited at a wavelength at emit light at another wavelength. The fluorescent molecules may be visible to the naked human eye. The fluorescent molecules may visible or identified via spectroscopic methods such to analyze the wavelength of light that are transmitted or absorbed by a solution comprising a fluorescent molecule.

The fluorescent molecules may have a distinct or known signature of excitation or emission wavelength of electromagnetic radiation. The detection of a fluorescent molecule signature may comprise identifying an amplitude or amplitudes of signal at different wavelengths. In some cases, the fluorescent molecule signature may comprise a signal at wavelengths that overlaps with wavelengths that may be generated by reagents in the chemical composition. In some cases, the excitation wavelength of the molecule may comprise a signal that overlaps with wavelengths that may be generated by reagents in the chemical composition. In some cases, the signals of the reaction and the fluorescent molecule may be simultaneously detected. Non-limiting examples of fluorescent molecules that may be used include Alexa Fluor 350, Alexa Fluor 350, Alexa Fluor 405, Alexa Fluor 488, Alexa Fluor 532, Alexa Fluor 546, Alexa Fluor 555, Alexa Fluor 568, Alexa Fluor 594, Alexa Fluor 647, Alexa Fluor 680, Alexa Fluor 750, Cy3, Cy5, Texas Red, Fluorescein (FITC), 6-FAM, 5-FAM, HEX, JOE, TAMRA, ROX, BODIPY FL, Pacific Blue, Pacific Green, Coumarin, Oregon Green, Pacific Orange, Trimethylrhodamine (TRITC), DAPI, APC, Cyan Fluorescent Protein (CFP), Green Fluorescent Protein (GFP), Red Fluorescent Protein (RFP), Phycoerythin (PE), quantum dots (for example, Qdot 525, Qdot 565, Qdot 605, Qdot 705, Qdot 800), or derivatives thereof.

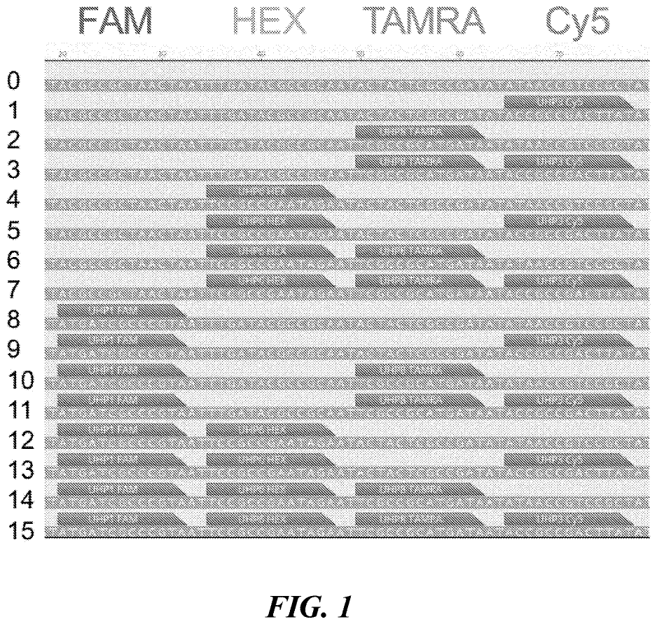

FIG. 1 shows an example schematic of different sequences and oligonucleotide probe binding sites. Example fluorophores are listed and each row lists a sequence that can bind to specific probes. A block is shown above the sequence demonstrating a probe that can bind to that sequence. For example, row Ishows a probe binding that is labeled with Cy5 and can bind to a Cy5 probe binding site. The example probe binding regions can be part of a longer oligonucleotides such as a tailed primer or molecular inversion probe as described elsewhere herein. For example, a tailed primer may comprise a target specific region and the sequence of row 2. When the target is present in the sample, the tailed primer may bind to the target and, upon extension of the primer, may generate a nucleic acid comprising the TAMRA probe binding site. Upon addition of the probes and extension of the nucleic acid, a TAMRA signal may be generated allowing for detection of the target. The example sequences also demonstrate the use of filler sequences or sequences that do not bind to a probe. For example, row 1 shows a sequence that has three filler sequences that do not bind to a universal FAM probe, a universal HEX probe, or a universal TAMRA probe, while having a sequence that does bind to the universal Cy5 probe. In comparison, row 7 has one filler sequence that does not bind to a FAM probe with three probe binding sites that bind to a universal HEX probe, a universal TAMRA probe, and a universal Cy5 probe. These two rows have a sequences of similar length, yet bind different universal probes. Different barcode sequence that bind different combination of probes may be constructed by using different combinations of filler sequences and probe binding sequences.

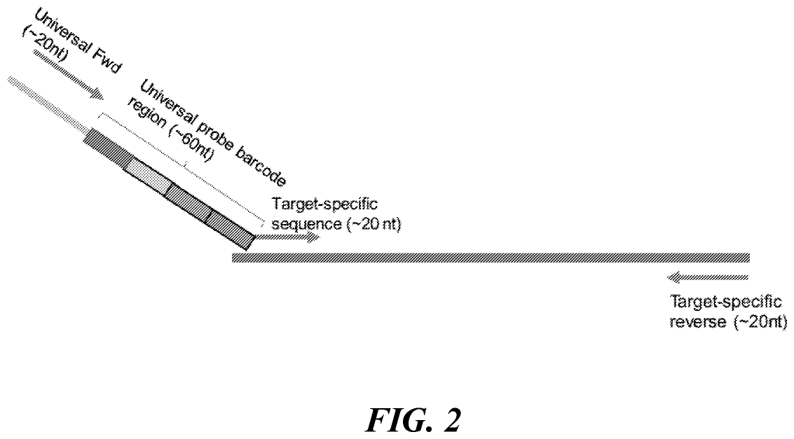

FIG. 2 shows an example schematic of the methods and oligonucleotides of the disclosure. An analyte can be amplified or extended using a oligonucleotide with a “target specific sequence” and a “universal probe barcode,” which comprises multiple probe binding sites. The example oligonucleotide depicted in FIG. 2, show four different sites, similar to as shown in FIG. 1, where each site may comprise a sequence that is able (or is unable) to bind to specific universal probe. A reverse primer can amplify this to generate an probe binding nucleic acid. A universal forward primer can amplify or extend this probe binding nucleic acid. As illustrated in the figure, the example probe binding barcode comprises 4 sections, which can correlate to the 4 sections illustrated in FIG. 1 corresponding to universal FAM, HEX, TAMRA, and Cy5.

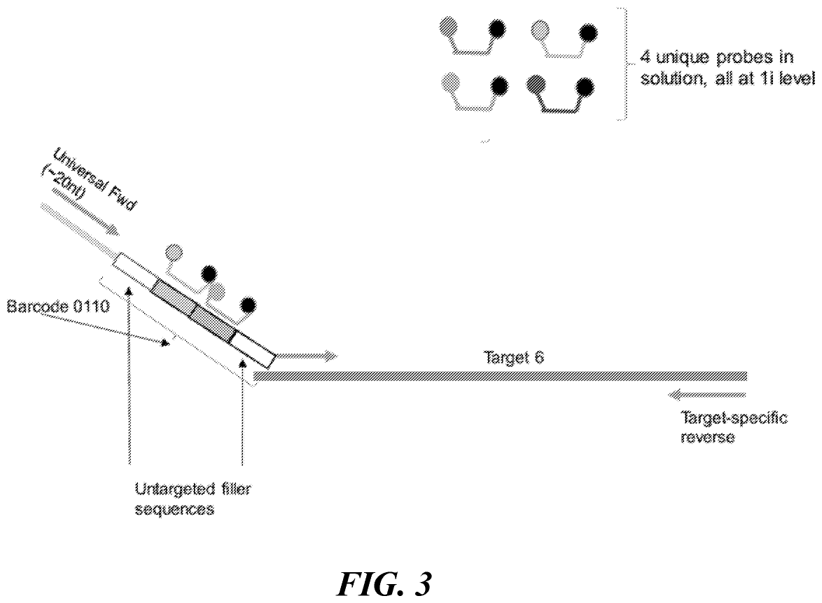

FIG. 3 shows an example schematic of the methods and oligonucleotides of the disclosures including a set of probes. An analyte can be amplified or extended using a oligonucleotide with a “target specific sequence” and a “universal probe barcode. A reverse primer can amplify this to generate an probe binding nucleic acid. Probe in solution can bind to the probe binding region and via amplification or extension of the probe binding nucleic acid via the universal primer, a signal can be generated that corresponds to the probe binding sites. FIG. 3 shows an example of a 0110 barcode. This numeration system has 4 places, each indicating the presence of a signal at a given site. The barcode generates no signal in a first channel, a signal of intensity of 1 (or i) in a second channel, a signal of intensity of 1 (ori) in a third channel, and no signal in a fourth channel. As described elsewhere, the probe binding region comprises filler sequences so that no probe binding occurs at certain regions and only probes corresponding to the second and third channel generate a signal. FIG. 3 shows an example oligonucleotide the corresponds to row 6 of FIG. 1, and generates a HEX and TAMRA signal

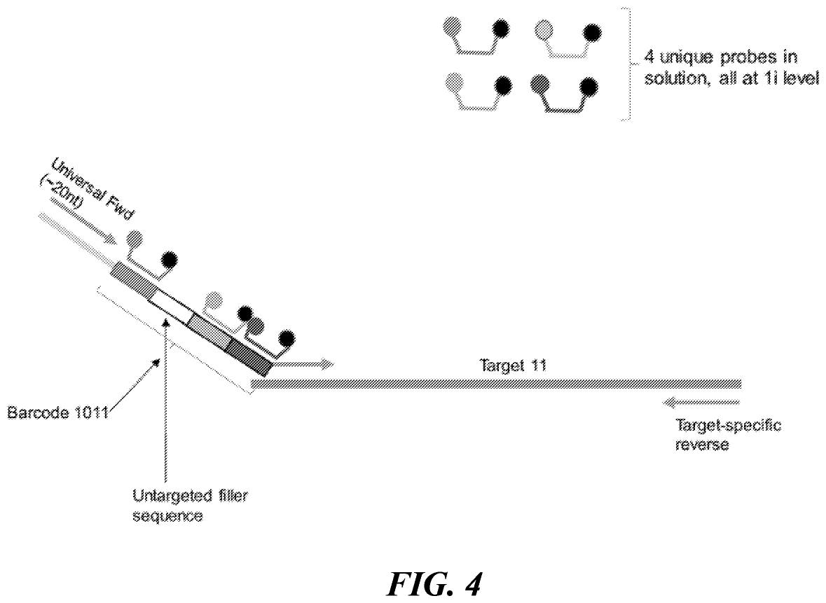

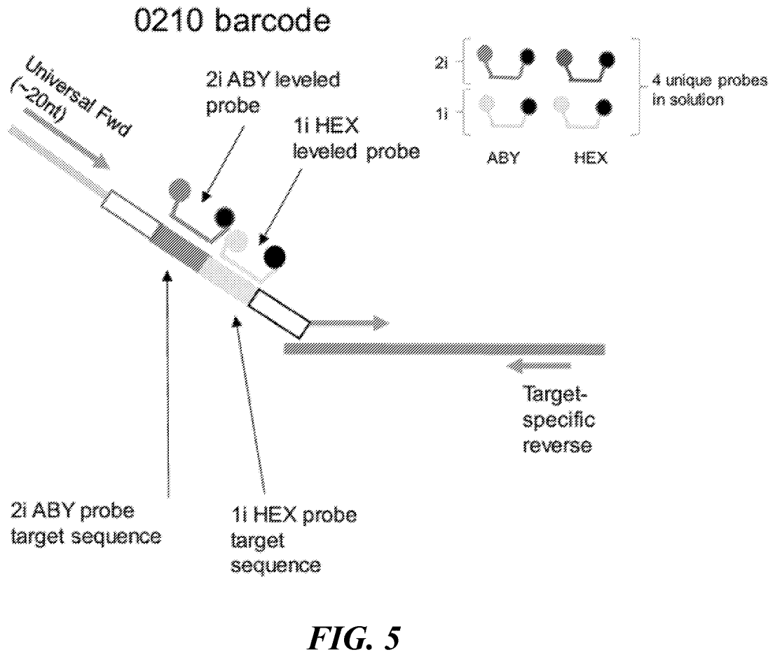

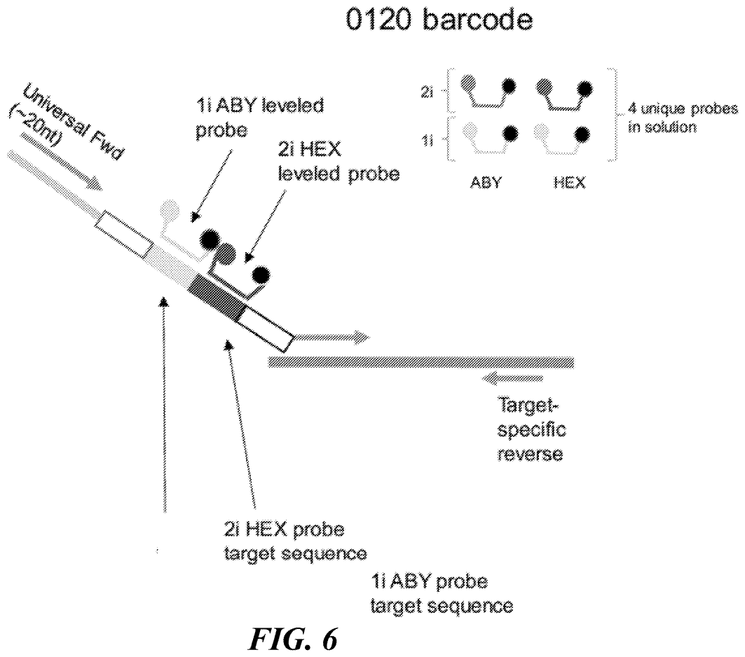

FIG. 4 shows an example schematic of the methods and oligonucleotides of the disclosure. FIG. 4 shows a similar schematic to FIG. 3 with a different probe binding site region and results in different probes binding. FIG. 4 shows an example of a 1011 barcode and corresponds to row 11 of FIG. 1. FIG. 5 shows similar schematic to FIG. 4, with a probe that generate a 2i signal. The primer shown in FIG. 5 shows an example 0210 barcode. The primer comprises a probe binding region comprising a probe binding site that binds to a 2i ABY probe and a probe binding site that binds to a 1i HEX probe. FIG. 6 shows a similar schematic to FIG. 5, with a different probe that generates a 2i signal, and represents an example 0120 barcode with a 1i ABY probe and a 2i HEX probe.

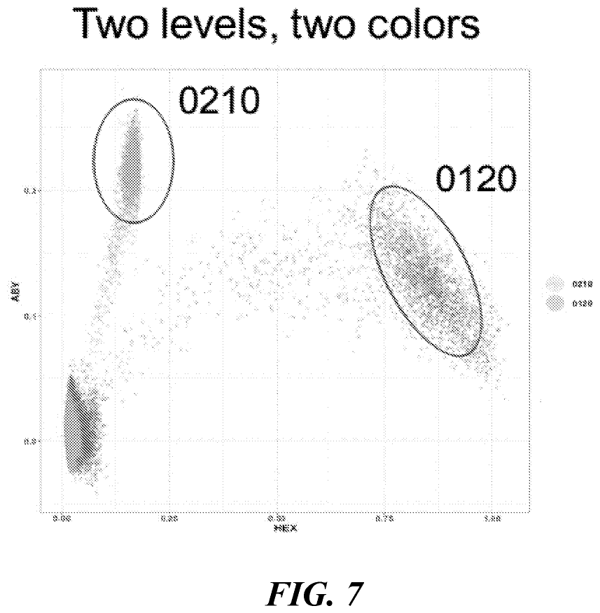

FIG. 7 shows data demonstrating distinct populations of analytes that are subjected to the methods of the disclosure. Two distinct populations are generated and encircled demonstrating the ability to distinguish between different analytes. A first population shows partitions comprising a first analyte that has been encoded by a 0210 barcode, where as second population shows partitions comprising a different analyte that is encoded by a 0120 barcode.

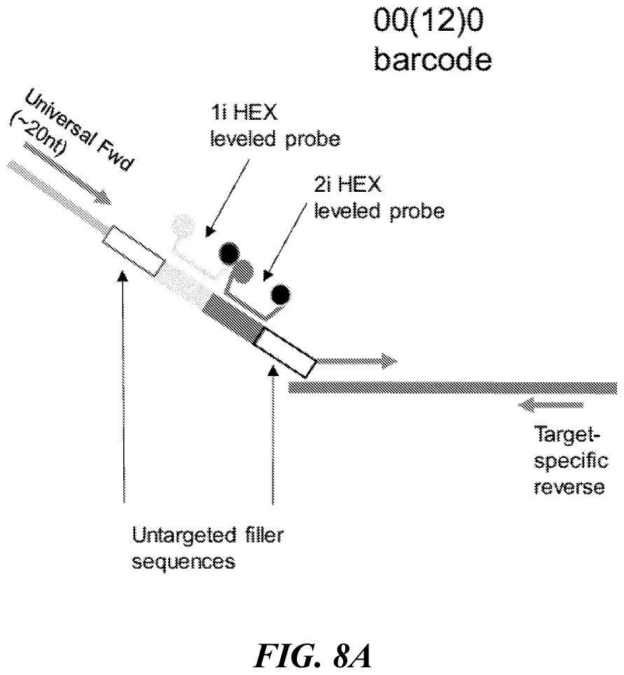

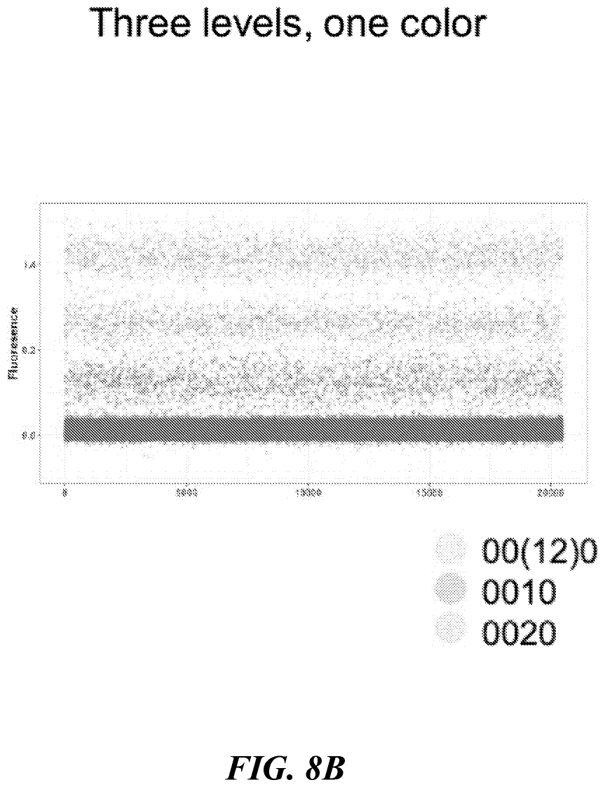

FIG. 8A shows an example schematic of the methods and oligonucleotides of the disclosure including a set of probes. A probe able to generate a 2i signal and a probe able to generate a 1i signal of the fluorophore is shown. This signal generated can be cumulative and result in a 3i signal. FIG. 8A show probe binding region comprising a site that bind to a 2i HEX probe and a site that binds to a 1i HEX probe. FIG. 8B shows data demonstrating detection of 3 distinct intensity levels. Analytes are resolved based on intensity levels in same color channel. Three different levels can be generated as follows: (i) A probe of 1i, (ii) a probe of 2i, and (iii) a combination of a probe of 1i and 2i, such as shown in FIG. 8A.

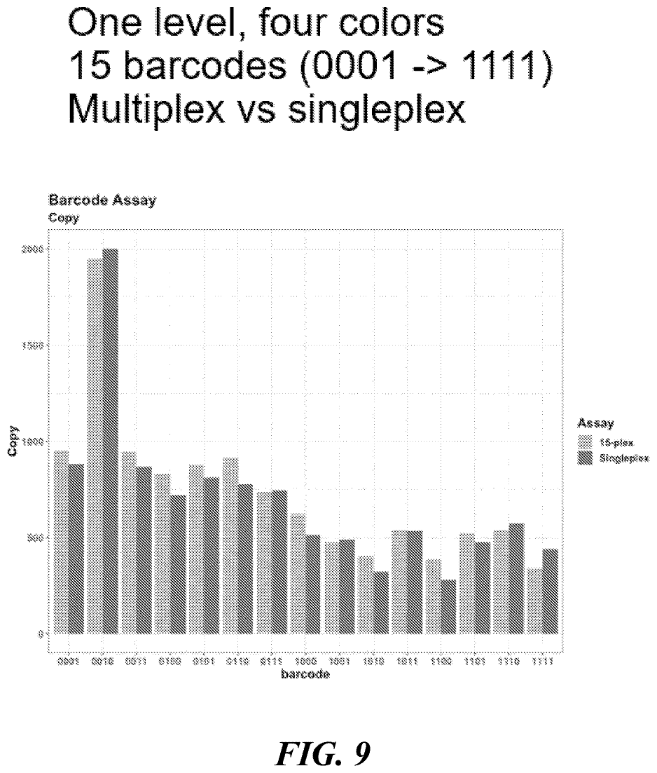

FIG. 9 shows data demonstrating detection of different analytes with different barcodes in multiplex versus single plex. FIG. 9 demonstrates that multiplexing these different analytes does not adversely affect analysis and that the barcodes can be used in multiplexed reactions.

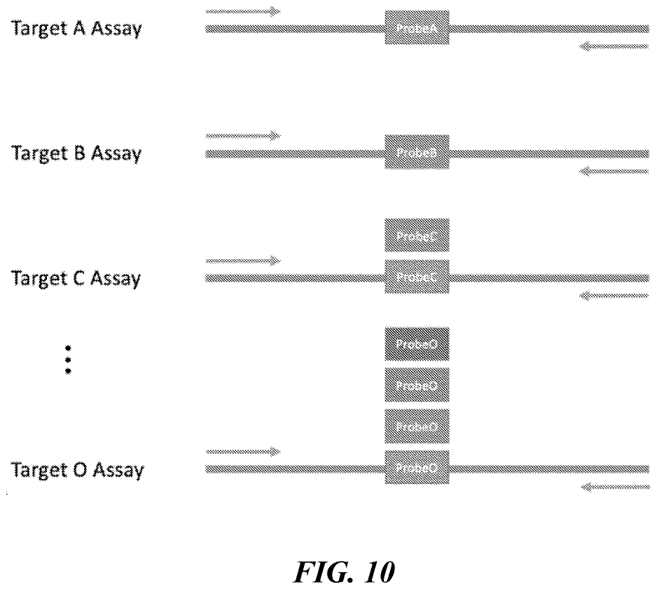

FIG. 10 shows an example schematic for another method of detection of different analytes. Each analyte can be encoded have one or more different probes that generate different colors. Different probes that have a same sequence can have different fluorophores. As shown, Target A is detected via one type of probe that generate Color 1. Target B is detected via one type of probe that generates Color 2. Target C is detected via two types of probe, one that generates Color 1 and one that generates Color 2. Target C is detected via 4 types of probes, one that generates Color 1, one that generates Color 2, one that generates Color 3, and one that generates Color 4. Similar to as shown for universal barcodes, different analytes may be encoded using different combinations of colors and intensities using probes of a same target specific sequence and with different appended signal tags or fluorophores.

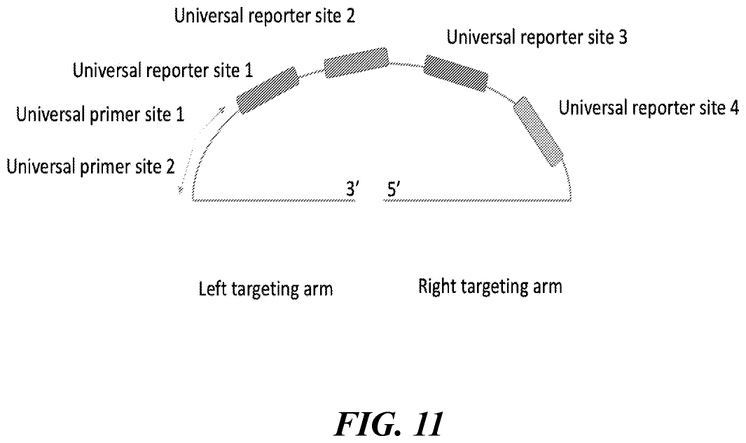

FIG. 11 shows an example oligonucleotide of the disclosure. As depicted, the oligonucleotide comprises a left targeting arm at a first end that can anneal to a target and a right targeting arm at a second end that can anneal to a target at a sequence different that the sequence that the left targeting arm anneals to. In between the two targeting arms can be multiple reporter sites (e.g., one or more probe binding sites) labeled as universal reporter site 1, universal reporter site 2, universal reporter site 3, universal reporter site 4. These universal reporter sites can be analogous to those shown in FIGS. 1-6 and 8A, and may be able to bind to different probes and generate signals of a particular intensity. The universal reporter sites can be replaced or otherwise be a filler sequence such as those described elsewhere herein. The oligonucleotide may comprise universal primer sequences that allow for replication of the probe and allow for detection of the probes.

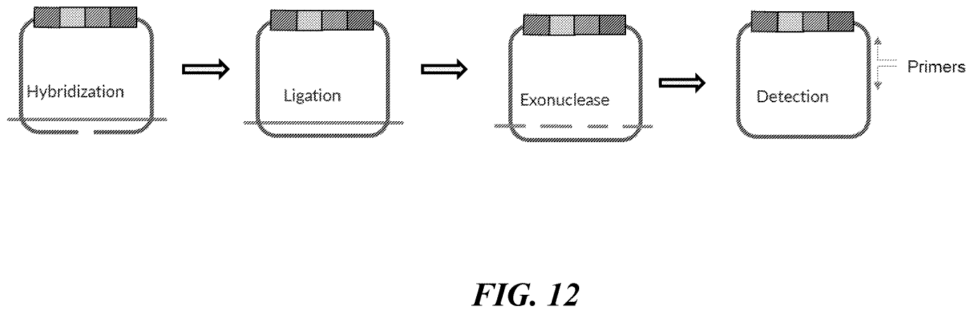

FIG. 12 shows a schematic of an example workflow using oligonucleotides of the disclosure. Using oligonucleotides such as one shown in FIG. 11, the oligonucleotides may hybridize to a target After hybridization, the two ends of the probe may be ligated together. The ends of the probes may initially be directly adjacent to one another and be able to be ligated. Alternatively the ends may anneal such that the ends are one or more nucleotides away. The ends may be subjected to a gap fill reaction (not shown) which adds additional nucleotides to one or both ends such that the resulting oligonucleotide has adjacent ends, and then can be ligated together. The oligonucleotides may be subjected to an exonuclease reaction to degrade any target nucleic acids or non-circularized probes, such as those that failed to annal to a target and were not ligated. The remaining probes may then be subjected to an amplification or extension reaction to allow for detection. Detection may occur be adding in probes that bind to the probe binding regions. These probes may be degraded via the amplification or extension reaction thereby generating a detectable signal. The circularized probes may optionally be linearized or cleaved prior to detection (not shown). A cleavage reaction may allow for the generation of a linear nucleic acid (as opposed to a circularized nucleic acid), which may allow for different types of reaction to be performed.

### Digital Assays

In some aspects, the present disclosure provides methods for performing a digital assay. A method for performing a digital assay may comprise partitioning a plurality of nucleic acid targets and a plurality of oligonucleotide probes into a plurality of partitions. In some cases, two, three, four, five, or more nucleic acid targets may be partitioned into a plurality of partitions together with two, three, four, five, six, seven, eight, nine, ten, 11, 12, 13, 14, 15, 16, 17, 18, 19, 20 or more oligonucleotide probes. Following partitioning, the nucleic acid targets may be amplified in the partitions, for example, by polymerase chain reaction (PCR). Next, N signals may be generated from the oligonucleotide probes. Each signal of the N signals may correspond to the presence of a unique combination of nucleic acid targets in a partition. Following signal generation, the N signals may be detected in a single optical channel. The signals may be detected using, for example, fluorescence detection in a single-color channel.

A method for performing a digital assay may comprise amplifying nucleic acid targets derived from a sample in a plurality of partitions comprising oligonucleotide probes complementary to one or more regions of nucleic acid targets. Each oligonucleotide probe may be labeled with a fluorophore. The fluorophores may be capable of being detected in a single optical channel. For example, the fluorophores may each comprise similar emission wavelength spectra, such that they can be detected in a single optical channel. Following partitioning, N signals may be detected from the plurality of partitions if one or more of the nucleic acid targets is present. Each of the N signals may correspond to a unique combination of one or more of the nucleic acid targets present in a partition. From the N signals, the presence or absence of each of the nucleic acid targets in the sample may be determined.

At least one signal of the plurality of signals may correspond with the presence of a unique combination of two or more of the first or second pluralities of nucleic acid molecules in a single partition. For example, one signal may correspond to the presence of two nucleic acid molecules (e.g., two copies of a nucleic acid sequence) in a single droplet. A signal of the plurality of signals may correspond with two or more unique combinations of the first or second pluralities of nucleic acid molecules in a single partition (e.g., may be an ambiguous signal). For example, a signal may correspond with the presence of one nucleic acid molecule and may also correspond with the presence of two nucleic acid molecules.

A reaction may comprise generating a cumulative signal measurement. Assays of the present disclosure may comprise comparing two or more cumulative signal measurements to unambiguously detect any combination of nucleic acid targets in a sample. A cumulative signal measurement may comprise one or more signals generated from one or more probes provided to a sample solution. A cumulative signal measurement may be a signal intensity level which corresponds to the sum of signals generated from multiple oligonucleotide probes. For example, two probes may each bind to a nucleic acid molecule, where each probe generates a signal of a given wavelength at 1× intensity. Measurement of these signals would generate a cumulative signal measurement corresponding to the sum of both signal intensities, namely a 2× signal intensity.

Methods of the present disclosure may comprise partitioning a sample or mixture into a plurality of partitions. A sample of mixture may comprise nucleic acids, oligonucleotide probes, and/or additional reagents into a plurality of partitions. A partition may be a droplet (e.g., a droplet in an emulsion). A partition may be a microdroplet. A partition may be a well. A partition may be a microwell. Partitioning may be performed using a microfluidic device. In some cases, partitioning is performed using a droplet generator. Partitioning may comprise dividing a sample or mixture into water-in-oil droplets. A droplet may comprise one or more nucleic acids. A droplet may comprise a single nucleic acid. A droplet may comprise two or more nucleic acids. A droplet may comprise no nucleic acids. Each droplet of a plurality of droplets may generate a signal. A plurality of signals may comprise the signal(s) generated from each of a plurality of droplets comprising a subset of a sample.

### Nucleic Acid Targets or Nucleic Acid Analytes

A nucleic acid target or nucleic acid analyte of the present disclosure may be derived from a sample. A biological sample may be a sample derived from a subject. A sample may comprise any number of macromolecules, for example, cellular macromolecules. A sample may comprise a plurality of cells. A sample may be a tissue sample, such as a biopsy, core biopsy, needle aspirate, or fine needle aspirate. The sample may be a tumor sample. A sample may be a fluid sample, such as a blood sample, plasma sample, urine sample, or saliva sample. A sample may be a skin sample. A biological sample may be a cheek swab. A sample may be a plasma or serum sample. A sample may comprise one or more cells. The one or more cells may be derived from a tumor. A biological sample may be, for example, blood, plasma, serum, urine, saliva, mucosal excretions, sputum, stool or tears. The sample may be obtained or derived from an environmental sample. For example, the sample may be a water sample or soil sample, or other samples found outside of a subject's body. The sample may be a wastewater sample. The sample may be a collection of samples. For example, a sample may be pooled with other sample and then subjected to methods described elsewhere herein.

A nucleic acid target may be derived from one or more cells. A nucleic acid target may comprise deoxyribonucleic acid (DNA). DNA may be any kind of DNA, including genomic DNA. A nucleic acid target may be viral DNA. A nucleic acid target may comprise ribonucleic acid (RNA). RNA may be any kind of RNA, including messenger RNA, transfer RNA, ribosomal RNA, and microRNA. RNA may be viral RNA. The nucleic acids may comprise a human genomic sequence. The nucleic acids may comprise an animal genomic sequence. The nucleic acids may comprise a plant genomic sequence. The nucleic acids may comprise a fungal genomic sequence. The nucleic acids may comprise an archaeal genomic sequence. The nucleic acids may comprise a pathogen associated sequence. The nucleic acid may comprise a wild type sequence. The nucleic acid may comprise a variant sequence.

Nucleic acid targets may comprise one or more members. A member may be any region of a nucleic acid target. A member may be of any length. A member may be, for example, up to 1, 2, 3, 4, 5, 10, 20, 50, 100, 500, 1000, 5000, 10000, 50000, or 100000 nucleotides, or more. In some instances, a member may be a gene. A nucleic acid target may comprise a gene whose detection may be useful in diagnosing one or more diseases. The disease may be cancer. A gene may be a viral gene or bacterial gene whose detection may be useful in identifying the presence or absence of a pathogen in a subject. In some cases, the methods of the present disclosure are useful in detecting the presence or absence or one or more infectious agents (e.g., viruses, bacteria, fungi) in a subject. The nucleic acid targets may be a human gene. The nucleic acid targets may be associated with a disease, such as cancer. The nucleic acid target may be a nucleic acid derived from an infectious agent. For example, the nucleic acid target may comprise a sequence of an influenza gene. The nucleic acid target may allow a genotype to be determined. The nucleic acid target may be a region of the human genome that indicates a predisposition for a particular disease. For example, a particular mutation or SNP of in a subject may be associated with an increased risk of infection of a particular pathogen. For example, the detection of both a pathogenic nucleic acid sequence and the presence of the mutation in the subject's genome may indicate the subject is at a high risk.

The detection of nucleic acids sequences may indicate a disease state, disorder, or the presence of a pathogen. Markers for a predisposition for a particular disease may be detected by assays as disclosed elsewhere herein. The markers for predisposition may be related to other existing conditions, for example, another disease such as diabetes, cancer, heart disease, or a condition that results in the individual being immune compromised. The markers may be related to immune response, for example, markers that indicate an intensity of a response to an antigen. The markers may be related to specific mutations or SNPs that are associated with higher infections. For example, a SNP of a receptor may have a higher affinity to a virus, thereby allowing the virus to more easily recognize and infect a cell.

Nucleic acid targets may be of various concentrations in the reaction. The nucleic acid sample may be diluted or concentrated to achieve different concentrations of nucleic acids. The concentration of the nucleic acids in the nucleic acid sample may at least 0.1 nanograms per microliter (ng/μL), 0.2 ng/μL, 0.5 ng/μL, 1 ng/μL, 2 ng/μL, 3 ng/μL, 5 ng/μL, 10 ng/μL, 20 ng/μL, 30 ng/μL, 40, ng/μL, 50 ng/μL, 100 ng/μL, 1000 ng/μL, 10000 ng/μL or more. In some cases, the concentration of the nucleic acids in the nucleic acid sample may be at most ng/μL, 0.2 ng/μL, 0.5 ng/μL, 1 ng/μL, 2 ng/μL, 3 ng/μL, 5 ng/μL, 10 ng/μL, 20 ng/μL, 30 ng/μL, 40, ng/μL, 50 ng/μL, 100 ng/μL, 1000 ng/μL, 10000 ng/μL or less.

The nucleic analytes may comprise mutations, such as single nucleotides variations, and the methods of the disclosure may be able to distinguish between analytes that differ by one or more nucleotides. For example, a first analyte may generate a first set of signals and a second analyte may generate a second set of signals, wherein the first analyte and second analyte differ by one nucleotide. The ability to distinguish two analytes may based at least on the sequences of the oligonucleotides. For example, the oligonucleotides may be specific to an single nucleotide variant. For an molecular inversion probe-like oligonucleotide, the sequence at the end of the oligonucleotide may be single nucleotide specific or may be adjacent to the single nucleotide variant and detect the presence of the addition of a specific base. The ability to distinguish two analyte may be based on the presence of blocking groups. For example, a blocking group may be present that can be cleaved by an enzyme when a perfect duplex is formed and unable to be cleaved when a mismatch is present.

### Sample Processing

A sample may be processed concurrently with, prior to, or subsequent to the methods of the present disclosure. A sample may be processed to purify or enrich for nucleic acids (e.g., to purify nucleic acids from a plasma sample). A sample comprising nucleic acids may be processed to purity or enrich for nucleic acid of interest. A sample may undergo an extraction to extract molecules used in the assay. For example, the extraction may use a column to bind or interact with a molecule. For example, an RNA extraction kit may be used such as a Qiagen RNA mini kit to extract or isolate RNA. A sample may be diluted. A sample may be diluted at least at 1:1, 1:2, 1:3, 1:4, 1:5, 1:6, 1:7, 1:8, 1:9, 1:10, 1:11, 1:12, 1:13, 1:14, 1:15; 1:16: 1:17, 1:18, 1:19, 1:20, 1:30, 1:40, 1:50, 1:60, 1:70, 1:80, 1:90, 1:100, or 1:1000, 1:10000, 1:100000 or more. A sample may be diluted at no more than at 1:1, 1:2, 1:3, 1:4, 1:5, 1:6, 1:7, 1:8, 1:9, 1:10, 1:11, 1:12, 1:13, 1:14, 1:15: 1:16: 1:17, 1:18, 1:19, 1:20, 1:30, 1:40, 1:50, 1:60, 1:70, 1:80, 1:90, 1:100, or 1:1000, 1:0000, 1:100000, or less. The sample may be diluted in a buffer or a solution. For example, the sample may be diluted in Tris-Ethylenediaminetetraacetic acid (TE) buffer. The sample may be diluted with a solution comprising alcohol. The sample may be diluted with a solution comprising sodium acetate.

### Nucleic Acid Enzymes

Mixtures and compositions of the present disclosure may comprise one or more nucleic acid enzymes. A nucleic acid enzyme may have exonuclease activity. A nucleic acid enzyme may have endonuclease activity. A nucleic acid enzyme may have RNase activity. A nucleic acid enzyme may be capable of degrading a nucleic acid comprising one or more ribonucleotide bases. A nucleic acid enzyme may be, for example, RNase H or RNase III. An RNase III may be, for example, Dicer. A nucleic acid may be an endonuclease I such as, for example, a T7 endonuclease I. A nucleic acid enzyme may be capable of degrading a nucleic acid comprising a non-natural nucleotide. A nucleic acid enzyme may be an endonuclease V such as, for example, an E. coli endonuclease V.

A nucleic acid enzyme may be a polymerase (e.g., a DNA polymerase). A DNA polymerase may be used. Any suitable DNA polymerase may be used, including commercially available DNA polymerases. A DNA polymerase generally refers to an enzyme that is capable of incorporating nucleotides to a strand of DNA in a template bound fashion. A polymerase may be Taq polymerase or a variant thereof. Non-limiting examples of DNA polymerases include Taq polymerase, Tth polymerase, Tli polymerase, Pfu polymerase, VENT polymerase, DEEPVENT polymerase, EX-Taq polymerase, LA-Taq polymerase, Expand polymerases, Sso polymerase, Poc polymerase, Pab polymerase, Mth polymerase, Pho polymerase, ES4 polymerase, Tru polymerase, Tac polymerase, Tne polymerase, Tma polymerase, Tih polymerase, Tfi polymerase, Platinum Taq polymerases, Hi-Fi polymerase, Tbr polymerase, Tfl polymerase, Pfutubo polymerase, Pyrobest polymerase, Pwo polymerase, KOD polymerase, Bst polymerase, Sac polymerase, Klenow fragment, and variants, modified products and derivatives thereof. For certain Hot Start Polymerase, a denaturation step at 94° C.-95° C. for 2 minutes to 10 minutes may be required, which may change the thermal profile based on different polymerases. A nucleic acid enzyme may be capable, under appropriate conditions, of degrading an oligonucleotide probe. For example, a nucleic acid enzyme may be a polymerase and comprise exo activity and degrade a probe resulting in a detectable signal. A nucleic acid enzyme may be capable, under appropriate conditions, of releasing a quencher from an oligonucleotide probe.

### Reactions

In various aspects disclosed elsewhere herein, reactions are performed. A reaction may comprise contacting nucleic acid targets with one or more oligonucleotide probes. A reaction may comprise contacting a sample solution volume (e.g., a droplet, well, tube, etc.) with a plurality of oligonucleotide probes, each corresponding to one of a plurality of nucleic acid targets, to generate a plurality of signals generated from the plurality of oligonucleotide probes. A reaction may comprise polymerase chain reaction (PCR).

In some aspects, the methods may comprise circularization reactions. Molecular inversion probes, or oligonucleotides with similar structures may be used such that probes may present in a configuration, upon annealing to a target, that may allow for the two ends of the probes to be connected. For example, the two ends may be directly adjacent to one another, and a ligation reaction may join the two ends together to generate a circularized nucleic acid. The two ends may be more than one nucleotide away from each other and may be subjected to gap fill reactions, such as polymerization reactions, extension reactions, or other reactions that attach additional nucleotides to the ends of the oligonucleotides. The gap fill reactions may then be followed by a ligation reaction to circularize the nucleic acid.

In some aspects, the methods may comprise exonuclease, cleavage, or nucleic acid degradation reactions. An exonuclease may be used to selectively degrade certain nucleic acids. For example, probes may be added to a mixture and allowed to anneal to targets and the probes may be circularized. Probes that did not anneal to a target may remain linear. An exonuclease may be used to selectively remove the linear probes that did not bind to any target, leaving the probes that bound to targets intact. The remaining probes may be subjected to additional reactions such to generate signals associated with the target and allow for detection of targets. Similarly, enzymes may selectively cleave at a base type or sequence. For example, the probe may be circularized and subjected to a exonuclease reaction that degrades non-circularized oligonucleotides (e.g., probes that did not anneal to a target). The circularized probe may then be subjected to a cleavage reaction to linearize the circularized probe and allow for additional reaction to be performed on the probes. The cleavage reaction may use a restriction enzyme that recognizes specific sequences. A cleavage reaction may use a enzyme that recognizes a specific base, such as a uracil-DNA glycosylase, that cleaves at a uracil base.

In some aspects, the disclosed methods comprise nucleic acid amplification. Amplification conditions may comprise thermal cycling conditions, including temperature and length in time of each thermal cycle. The use of particular amplification conditions may serve to modify the signal intensity of a signal, thereby enabling a signal (or plurality of signals) to correspond to a unique combination of nucleic acid targets. Amplification may comprise using enzymes such to produce additional copies of a nucleic. The amplification reaction may comprise using oligonucleotide primers as described elsewhere herein. The oligonucleotide primers may use specific sequences to amplify a specific sequence. The oligonucleotide primers may amplify a specific sequence by hybridizing to a sequence upstream and downstream of the primers and result in amplifying the sequence inclusively between the upstream and downstream primer. The oligonucleotide may be able to amplify more than one sequence analyte by hybridizing upstream or downstream of multiple different sequences. The amplification reaction may comprise the use of nucleotide tri-phosphate reagents. The nucleotide tri-phosphate reagents may comprise using deoxyribo-nucleotide tri-phosphate (dNTPs). The nucleotide tri-phosphate reagents may be used as precursors to the amplified nucleic acids. The amplification reaction may comprise using oligonucleotide probes as described elsewhere herein. The amplification reaction may comprise using enzymes. Non-limiting examples of enzymes include thermostable enzymes, DNA polymerases, RNA polymerases, and reverse transcriptases. The amplification reaction may comprise generating nucleic acid molecules of a different nucleotide types. For example, a target nucleic acid may comprise DNA and an RNA molecule may be generated. In another example, an RNA molecule may be subjected to an amplification reaction and a cDNA molecule may be generated.

### Thermal Cycling

Methods of the present disclosure may comprise thermal cycling. Thermal cycling may comprise one or more thermal cycles. Thermally cycling may be performed under reaction conditions appropriate to amplify a template nucleic acid with PCR. Amplification of a template nucleic acid may require binding or annealing of oligonucleotide primer(s) to the template nucleic acid. Appropriate reaction conditions may include appropriate temperature conditions, appropriate buffer conditions, and the presence of appropriate reagents. Appropriate temperature conditions may, in some cases, be such that each thermal cycle is performed at a desired annealing temperature. A desired annealing temperature may be sufficient for annealing of an oligonucleotide probe(s) to a nucleic acid target. Appropriate buffer conditions may, in some cases, be such that the appropriate salts are present in a buffer used during thermal cycling Appropriate salts may include magnesium salts, potassium salts, ammonium salts. Appropriate buffer conditions may be such that the appropriate salts are present in appropriate concentrations. Appropriate reagents for amplification of each member of a plurality of nucleic acid targets with PCR may include deoxyribonucleotide triphosphates (dNTPs). dNTPs may comprise natural or non-natural dNTPs including, for example, dATP, dCTP, dGTP, dTTP, dUTP, and variants thereof.

In various aspects, primer extension reactions are utilized to generate amplified product. Primer extension reactions generally comprise a cycle of incubating a reaction mixture at a denaturation temperature for a denaturation duration and incubating a reaction mixture at an elongation temperature for an elongation duration. In any of the various aspects, multiple cycles of a primer extension reaction can be conducted. Any suitable number of cycles may be conducted. For example, the number of cycles conducted may be less than about 100, 90, 80, 70, 60, 50, 40, 30, 20, 10, or 5 cycles. The number of cycles conducted may depend upon, for example, the number of cycles (e.g., cycle threshold value (Ct)) used to obtain a detectable amplified product (e.g., a detectable amount of amplified DNA product that is indicative of the presence of a target DNA in a nucleic acid sample). For example, the number of cycles used to obtain a detectable amplified product (e.g., a detectable amount of DNA product that is indicative of the presence of a target DNA in a nucleic acid sample) may be less than about or about 100 cycles, 75 cycles, 70 cycles, 65 cycles, 60 cycles, 55 cycles, 50 cycles, 40 cycles, 35 cycles, 30 cycles, 25 cycles, 20 cycles, 15 cycles, 10 cycles, or 5 cycles. Moreover, in some embodiments, a detectable amount of an amplifiable product (e.g., a detectable amount of DNA product that is indicative of the presence of a target DNA in a nucleic acid sample) may be obtained at a cycle threshold value (Ct) of less than 100, 75, 70, 65, 60, 55, 50, 45, 40, 35, 30, 25, 20, 15, 10, or 5.

The time for which an amplification reaction yields a detectable amount of amplified nucleic acid may vary depending upon the nucleic acid sample, the sequence of the target nucleic acid, the sequence of the primers, the particular nucleic acid amplification reactions conducted, and the particular number of cycles of the amplification, the temperature of the reaction, the pH of the reaction. For example, amplification of a target nucleic acid may yield a detectable amount of product indicative to the presence of the target nucleic acid at time period of 120 minutes or less; 90 minutes or less; 60 minutes or less; 50 minutes or less; 45 minutes or less; 40 minutes or less; 35 minutes or less; 30 minutes or less; 25 minutes or less; 20 minutes or less; 15 minutes or less; 10 minutes or less; or 5 minutes or less.

In some embodiments, amplification of a nucleic acid may yield a detectable amount of amplified DNA at time period of 120 minutes or less; 90 minutes or less; 60 minutes or less; 50 minutes or less; 45 minutes or less; 40 minutes or less; 35 minutes or less; 30 minutes or less; 25 minutes or less; 20 minutes or less; 15 minutes or less; 10 minutes or less; or 5 minutes or less.

### Oligonucleotide Primers

In various aspects disclosed elsewhere herein, oligonucleotide primers are used. An oligonucleotide primer (or “amplification oligomer”) of the present disclosure may be a deoxyribonucleic acid. An oligonucleotide primer may be a ribonucleic acid. An oligo nucleotide primer may comprise one or more non-natural nucleotides. A non-natural nucleotide may be, for example, deoxyinosine. The oligonucleotide primer may be able to hybridize to a first analyte and a second analyte, and may generates a first signal corresponding to said first analyte and a second signal corresponding to said second analytes.

A primer may comprise a first region complementary to an analyte and a second region comprising probe binding sites. The second region may comprise more than one probe binding sites. The second region may comprise more than two probe binding sites. The second region may comprise more than three probe binding sites. The second region may comprise more four probe binding sites. The probe binding sites may be the same or different compared to other primers in a reaction mixture. The primer may comprise combinations of probe binding sites that are different that the probe binding sites of other primers in a reaction mixture.

The primers may comprise additional regions that another primer can anneal to. For example, a primer can comprise an universal region that a universal primer can anneal to. A first rection using a primer comprising a universal region can anneal to a target and generate, via extension or amplification, a nucleic acid that comprise the target nucleic acid sequence and the universal region. In a second reaction, the universal primer may anneal to the universal region and generate additional copies of the nucleic acid. This may be especially advantageous for multiplexed workflows comprising multiple targets. For example, a mixture of different target specific primers that comprise universal regions can be used to amplify the multiple targets. As the resulting nucleic acids comprise universal regions, a universal primer can be used to amplify the multiple targets in a single reaction mixture, regardless of the original sequences of the targets.

As described elsewhere herein, amplification of targets can generate a signal via the degradation or removal of probes. Primers may comprise probe sites that may allow for targets to be labeled with probes sites via extension or amplification of the target specific primers. The probes may be allowed to anneal to the probe sites, and a second extension or amplification reaction may be performed to displace or degrade the probes, thereby generating a signal. This may be used in conjunction with primers with universal regions and the probe sites (and probes) may be 3′ to the universal region. The universal primers can then be used to generate the probe signal, and can allow for multiplexed generation of signals from multiple targets.

A primer may comprise filler sequences. For example, a primer may comprise a sequence that does not anneal to a target, probe, or another primer. The filler sequence may have low or no binding to other sequences in the mixture. The filler sequence may be used to generate different primers that have a same or similar length that perform different functions. For example, a first primer may comprise two different probe binding sites and second primer may comprise one probe binding site and a filler sequence. The first primer may be able to bind two probes and generate two different signals, whereas the second primer may anneal to only one probe a generate one signal. Using the filler sequences the primers may be of similar size may allow for improved multiplexing, for example, due to more similar melting temperatures or suitable reaction temperatures for the two primers.

An oligonucleotide primer may comprise a blocking group or blocking region. The blocking group be at a 3′ end of an oligonucleotide. A blocking group may be unextendible and may need to be cleaved to allow a primer to be extended. The blocking group may allow for primers to differentiate between different loci or alleles, for example, those with single nucleotide polymorphisms (SNPs). For example, a blocking group may be unextendible and may need to be cleaved by an enzyme. The enzyme may recognize a perfectly matched primer-target duplex, and may cleave the blocking group allowing for extension. A mismatched primer-target duplex may be unable to be recognized by the enzyme and fail to cleave off the blocking group, thereby blocking extension.

An oligonucleotide primer may be a forward primer. An oligonucleotide primer may be a reverse primer. An oligonucleotide primer may be between about 5 and about 50 nucleotides in length. An oligonucleotide primer may be at least 5, 10, 11, 12, 13, 14, 15, 16, 17, 18, 19, 20, 25, 30, 35, 40, 45, or 50 base pairs in length, or more. An oligonucleotide primer may be at most 50, 45, 40, 35, 30, 25, 20, 19, 18, 17, 16, 15, 14, 13, 12, 11, 10, or 5 nucleotides in length. An oligonucleotide primer may be about 5, 10, 11, 12, 13, 14, 15, 16, 17, 18, 19, 20, 25, 30, 35, 40, 45, or 50 base pairs in length.

A set of oligonucleotide primers may comprise paired oligonucleotide primers. Paired oligonucleotide primers may comprise a forward oligonucleotide primer and a reverse oligonucleotide primer. A forward oligonucleotide primer may be configured to hybridize to a first region (e.g., a 3′ end) of a nucleic acid sequence, and a reverse oligonucleotide primer may be configured to hybridize to a second region (e.g., a 5′ end) of the nucleic acid sequence, thereby being configured to amplify the nucleic acid sequence under conditions sufficient for nucleic acid amplification. Different sets of oligonucleotide primers may be configured to amplify different nucleic acid target sequences. For example, a first set of oligonucleotide primers may be configured to amplify a first nucleic acid sequence of a given length, and a second set of oligonucleotide primers may be configured to amplify a second nucleic acid sequence of shorter length than the first nucleic acid sequence. In another example, a first set of oligonucleotide primers may be configured to amplify a first nucleic acid sequence of a given length, and a second set of oligonucleotide primers may be configured to amplify a second nucleic acid sequence of longer length than the first nucleic acid sequence.

A mixture may comprise a plurality of forward oligonucleotide primers. A plurality of forward oligonucleotide primers may be a deoxyribonucleic acid. Alternatively, a plurality of forward oligonucleotide primers may be a ribonucleic acid. A plurality of forward oligonucleotide primers may be between about 5 and about 50 nucleotides in length. A plurality of forward oligonucleotide primer may be at least 5, 10, 11, 12, 13, 14, 15, 16, 17, 18, 19, 20, 25, 30, 35, 40, 45, or 50 base pairs in length, or more. A plurality of forward oligonucleotide primer may be at most 50, 45, 40, 35, 30, 25, 20, 19, 18, 17, 16, 15, 14, 13, 12, 11, 10, or 5 nucleotides in length.

A mixture may comprise a plurality of reverse oligonucleotide primers. A plurality of reverse oligonucleotide primers may be a deoxyribonucleic acid. Alternatively, a plurality of reverse oligonucleotide primers may be a ribonucleic acid. A plurality of reverse oligonucleotide primers may be between about 5 and about 50 nucleotides in length. A plurality of reverse oligonucleotide primer may be at least 5, 10, 11, 12, 13, 14, 15, 16, 17, 18, 19, 20, 25, 30, 35, 40, 45, or 50 base pairs in length, or more. A plurality of reverse oligonucleotide primer may be at most 50, 45, 40, 35, 30, 25, 20, 19, 18, 17, 16, 15, 14, 13, 12, 11, 10, or 5 nucleotides in length.

A set of oligonucleotide primers (e.g., a forward primer and a reverse primer) may be configured to amplify a nucleic acid sequence of a given length (e.g., may hybridize to regions of a nucleic acid sequence a given distance apart). A pair of oligonucleotide primers may be configured to amplify a nucleic acid sequence of a length of at least 50, at least 75, at least 100, at least 125, at least 150, at least 175, at least 200, at least 225, at least 250, at least 275, or at least 300 base pairs (bp), or more. A pair of oligonucleotide primers may be configured to amplify a nucleic acid sequence of a length of at most 300, at most 275, at most 250, at most 225, at most 200, at most 175, at most 150, at most 125, at most 100, at most 75, or at most 50 bp, or less.

In some aspects, the primer may be configured to hybridize, anneal or be homologous to sequences derived from humans. The sequence may be a sequence associated with cancer. The sequence may be associated with trisomy or fetal abnormalities. In some aspects, a mixture may include one or more synthetic (or otherwise generated to be different from the target of interest) primers for PCR reactions.

In some aspects, a mixture may be subjected to conditions sufficient to anneal an oligonucleotide primer to a nucleic acid molecule. In some aspects, a mixture may be subjected to conditions sufficient to anneal a plurality of oligonucleotide primers to a nucleic acid molecule.

In some aspects, a mixture may be subjected to conditions sufficient to anneal a plurality of oligonucleotide primers to a plurality of nucleic acid targets. The mixture may be subjected to conditions which are sufficient to denature nucleic acid molecules. Subjecting a mixture to conditions sufficient to anneal an oligonucleotide primer to a nucleic acid target may comprise thermally cycling the mixture under reaction conditions appropriate to amplify the nucleic acid target(s) with, for example, polymerase chain reaction (PCR).

Conditions may be such that an oligonucleotide primer pair (e.g., forward oligonucleotide primer and reverse oligonucleotide primer) are degraded by a nucleic acid enzyme. An oligonucleotide primer pair may be degraded by the exonuclease activity of a nucleic acid enzyme. An oligonucleotide primer pair may be degraded by the RNase activity of a nucleic acid enzyme. Degradation of the oligonucleotide primer pair may result in release of the oligonucleotide primer. Once released, the oligonucleotide primer pair may bind or anneal to a template nucleic acid.

### Oligonucleotide Probes

In various aspects disclosed elsewhere herein, oligonucleotide probes are used. Samples, mixtures, kits, and compositions of the present disclosure may comprise an oligonucleotide probe, also referenced herein as a “detection probe” or “probe”. An oligonucleotide probe may be a nucleic acid (e.g., DNA, RNA, etc.). An oligonucleotide probe may comprise a region complementary to a region of a nucleic acid target. The concentration of an oligonucleotide probe may be such that it is in excess relative to other components in a sample.

The oligonucleotide probe may be able to hybridize to a first analyte and a second analyte, and may generates a first signal corresponding to said first analyte and a second signal corresponding to said second analytes.

An oligonucleotide probe may comprise a non-target-hybridizing sequence. A non-target-hybridizing sequence may be a sequence which is not complementary to any region of a nucleic acid target sequence. An oligonucleotide probe comprising a non-target-hybridizing sequence may be a hairpin detection probe. An oligonucleotide probe comprising a non-target-hybridizing sequence may be a molecular beacon probe. Examples of molecular beacon probes are provided in, for example, U.S. Pat. No. 7,671,184, incorporated herein by reference in its entirety. An oligonucleotide probe comprising a non-target-hybridizing sequence may be a molecular torch. Examples of molecular torches are provided in, for example, U.S. Pat. No. 6,534,274, incorporated herein by reference in its entirety.

A sample may comprise more than one oligonucleotide probe. Multiple oligonucleotide probes may be the same or may be different. An oligonucleotide probe may be at least 5, at least 10, at least 15, at least 20, or at least 30 nucleotides in length, or more. An oligonucleotide probe may be at most 30, at most 20, at most 15, at most 10 or at most 5 nucleotides in length. In some examples, a mixture comprises a first oligonucleotide probe and one or more additional oligonucleotide probes. An oligonucleotide probe may be a nucleic acid (e.g., DNA, RNA, etc.). An oligonucleotide probe may be at least 2, 3, 4, 5, 6, 7, 8, 9, 10, 20, 30, 40, or 50 nucleotides in length, or more. An oligonucleotide probe may be at most 50, 40, 30, 20, 10, 9, 8, 7, 6, 5, 4, 3, or 2 nucleotides in length.

In some cases, 2, 3, 4, 5, 6, 7, 8, 9, 10, 11, 12, 13, 14, 15, 20, or more different oligonucleotide probes may be used. An oligonucleotide probe may comprise a signal tag. In some cases, each oligonucleotide probe in a plurality of oligonucleotide probes may comprise an identical fluorophore. In some cases, the oligonucleotide probe comprises an identical fluorophore to another oligonucleotide probe. In some cases, each oligonucleotide probe comprises a different fluorophore. In some case, each fluorophore is capable of being detected in a single optical channel. In other case, a fluorophore may be detected in multiple channels. In some cases, an oligonucleotide probe may have similar or the same detectable agent or fluorophore as another oligonucleotide probe in the sample. In some cases, an oligonucleotide probe may have a different detectable agent or fluorophore as compared to another oligonucleotide probe in the sample. In some cases, an oligonucleotide probe may have similar sequence or be capable or binding a similar sequence as another oligonucleotide probe in the sample. In some cases, an oligonucleotide probe may have a different sequence or be capable of binding a different sequence as compared to another oligonucleotide probe in the sample.

An oligonucleotide probe may comprise a detectable label. A detectable label may be a chemiluminescent label. A detectable label may comprise a fluorescent label. A detectable label may comprise a fluorophore. A fluorophore may be, for example, FAM, TET, HEX, JOE, Cy3, or Cy5. A fluorophore may be FAM. A fluorophore may be HEX. An oligonucleotide probe may further comprise one or more quenchers. A quencher may inhibit signal generation from a fluorophore. A quencher may be, for example, TAMRA, BHQ-1, BHQ-2, or Dabcy. A quencher may be BHQ-1. A quencher may be BHQ-2.

A probe may correspond to a region of a nucleic acid target. For example, a probe may have complementarity and/or homology to a region of a nucleic acid target. A probe may comprise a region which is complementary or homologous to a region of a nucleic acid target. A probe corresponding to a region of a nucleic acid target may be capable of binding to the region of the nucleic acid target under appropriate conditions (e.g., temperature conditions, buffer conditions, etc.). For example, a probe may be capable of binding to a region of a nucleic acid target under conditions appropriate for polymerase chain reaction. A probe may correspond to an oligonucleotide which corresponds to a nucleic acid target. For example, an oligonucleotide may be a primer with a region complementary to a nucleic acid target and a region complementary to a probe.

A oligonucleotide probe may be a molecular inversion probe, or comprise a structure similar to a molecular inversion probe. For example, a probe may comprise (i) a first region at a first end of the probe that anneals to a nucleic acid target and (ii) a second region at a second end of the probe that anneals to the nucleic acid target at a different sequence. The oligonucleotide probe, when annealed to the target, may be able to be circularized via additional reactions, and generate a circularized probe. The oligonucleotide may be able to anneal to other probes (e.g., Taqman probes) and may comprise one or more probe binding sites. For example, an oligonucleotide may comprise from 5′ to 3′ (i) first region complementary to an analyte and a second region comprising probe binding sites, and a third region complementary to the analyte at different sequence. The second region may comprise more than one probe binding sites. The second region may comprise more than two probe binding sites. The second region may comprise more than three probe binding sites. The second region may comprise more four probe binding sites. The probe binding sites may be the same or different compared to other oligonucleotides in a reaction mixture. The oligonucleotide may comprise combinations of probe binding sites that are different that the probe binding sites of other oligonucleotides in a reaction mixture.

A probe may comprise filler sequences. For example, a probe may comprise a sequence that does not anneal to a target, primer, or another probe. The filler sequence may have low or no binding to other sequences in the mixture. The filler sequence may be used to generate different probes that have a same or similar length that perform different functions. For example, a first probe may comprise two different probe binding sites and second probe may comprise one probe binding site and a filler sequence. The first probe may be able to bind two probes and generate two different signals, whereas the second primer may anneal to only one probe a generate one signal. Using the filler sequences the probe may be of similar size may allow for improved multiplexing, for example, due to more similar melting temperatures or suitable reaction temperatures for the two probes.

A probe may comprise a uracil or other base that can be selectively recognized by an enzyme. For example, probe may comprise a uracil and may be cleaved via recognition of a Uracil-DNA glycosylases (UDG). For example, the probe may be circularized and then subsequently cleaved by a UDG.

The probe may be a universal probe. The probe may be non-specific to a specific analyte and bind to a region that is present in multiple different nucleic acids. As described throughout the disclosure, nucleic acids may be generated that have probe binding sites, such as via extension of a tailed primer or circularization of molecular inversion probe. These probe binding site may be universal probe binding sites, such that the sequence is common across different nucleic acids. Thus the addition of a universal probe molecule can allow for binding to multiple different molecules and generating a signal from multiple molecules. For example, a FAM probe may have a set universal sequence. For a nucleic acid to generate a FAM signal, a sequence that binds to the FAM probe may be a part of the tail of the primer. When encoding or barcoding analytes, the primers or probe may be designed to use the universal probe binding sequences to generate the signal associated with that probe.

In some aspects, the probe may be configured to hybridize, anneal or be homologous to sequences derived from a virus. For example the virus may comprise an influenza virus, coronavirus, respiratory syncytial virus, hepatitis virus, herpesvirus, or papillomavirus. In some aspects, the probe may be configured to hybridize, anneal or be homologous to sequences derived from humans. The sequence may be a sequence associated with cancer. The sequence may be associated with trisomy or fetal abnormalities.

A probe may be provided at a specific concentration. In some cases, a second nucleic acid probe is provided at a concentration of at least about 2X, about 3X, about 4X, about 5×, about 6X, about 7X, about 8X, or more. In some cases, a second nucleic acid probe is provided at a concentration of at most about 8X, about 7X, about 6X, about 5X, about 4X, about 3X, or about 2X. In some cases, a second nucleic acid probe is provided at a concentration of about 2X, about 3X, about 4X, about 5X, about 6X, about 7X, or about 8X. X may be a concentration of a nucleic acid probe provided in the disclosed methods. In some cases, X is at least 50 nM, 100 nM, 150 nM, 200 nM, 250 nM, 300 nM, 350 nM, 400 nM, 450 nM, 500 nM, or greater. In some cases, X is at most 500 nM, 450 nM, 400 nM, 350 nM, 300 nM, 250 nM, 200 nM, 150 nM, 100 nM, or 50 nM. X may be any concentration of a nucleic acid probe.

A probe may be a nucleic acid complementary to a region of a given nucleic acid target. Each probe used in the methods and assays of the presence disclosure may comprise at least one fluorophore. A fluorophore may be selected from any number of fluorophores. A fluorophore may be selected from three, four, five, six, seven, eight, nine, or ten fluorophores, or more. One or more oligonucleotide probes used in a single reaction may comprise the same fluorophore. In some cases, all oligonucleotide probes used in a single reaction comprise the same fluorophore. Each probe may, when excited and contacted with its corresponding nucleic acid target, generate a signal. A signal may be a fluorescent signal. A plurality of signals may be generated from one or more probes.

An oligonucleotide probe may have less than 50%, 40%, 30%, 20%, 10%, 5%, or 1% complementarity to any member of a plurality of nucleic acid targets. An oligonucleotide probe may have no complementarity to any member of the plurality of nucleic acid targets.

### Signal Generation

Thermal cycling may be performed such that one or more oligonucleotide probes are degraded by a nucleic acid enzyme. An oligonucleotide probe may be degraded by the exonuclease activity of a nucleic acid enzyme. An oligonucleotide probe may generate a signal upon degradation. In some cases, an oligonucleotide probe may generate a signal only if at least one member of a plurality of nucleic acid targets is present in a mixture.

In various aspects, extension reactions and amplification reactions may be used to allow for the generation of signals. The extension reaction and amplification reaction may be used to generate a signal correspond to an analyte. The extension reaction may extend an oligonucleotide that can hybridize to more than one analyte. Based on the hybridization partner, the extension reaction may generate a different signal. Extension or amplification of a first analytes may generate a first signal whereas the extension or amplification of a second analyte may generate a second signal. The efficiency of the hybridization reactions may affect the extension reaction or the generation of a signal. The extension or amplification reaction may generate a signal by degrading or reaction with the oligonucleotide that can hybridize to more than one analyte. The oligonucleotide that can hybridize to more than one analyte may be a probe, and the extension or amplification reaction may allow generation of a signal from the probe. The probe may hybridize with different efficiency or affinity and may allow the generation of a different signal based on the analyte hybridized thereto.

Signal generation may correspond to reactions conditions of reactions relating to signal generation. The signal generation may be altered by a hybridization efficiency of the oligonucleotide. For example, an oligonucleotide may have a hybridization efficiency to a first analyte and a different hybridization efficiency to a second analyte which may in turn affect the generation of signal or alter the resulting signal that is generated. In the case of amplification or extension reactions, a time period or temperature may be altered such to change the signal generation efficiency or a kinetic signature shape. For example, a sample may comprise more than one analyte and an oligonucleotide that can hybridize to more than one analyte may be added to the sample. The different signal generation efficiency or kinetic shape of a reaction may be used to differentiate a first analyte and a second analyte. The annealing temperature of a reaction may be altered such that the hybridization to one analyte is favored over the hybridization to another analyte. Multiple reactions may be performed at different annealing temperature (for example using a gradient) that allows for a signal to be generated and distinguishable for different analytes. The reactions may be performed such that a first reaction has a more stringent annealing condition compared to a second reaction. The reactions may comprise an annealing time, and the annealing time may be modulated to affect the generation of a signal. For example, a first reaction may comprise an annealing time that is longer than an annealing time for a second reaction. For example, a first reaction may comprise an annealing time that is shorter than an annealing time for a second reaction. Similarly, extension times and temperatures may be modulated to affect the generation of a signal and allow different signal to be obtained based on the analyte. For example, a first reaction may comprise an extension time that is longer than an extension time for a second reaction. For example, a first reaction may comprise an extension time that is shorter than an extension time for a second reaction. For example, a first reaction may comprise an extension temperature that is higher (or lower) than an extension temperature for a second reaction. For example, a first reaction may comprise an extension temperature that is lower than an extension temperature for a second reaction.

A reaction may generate one or more signals. A reaction may generate a cumulative intensity signal comprising a sum of multiple signals. A signal may be a chemiluminescent signal. A signal may be a fluorescent signal. A signal may be generated by an oligonucleotide probe. For example, excitation of a hybridization probe comprising a luminescent signal tag may generate a signal. A signal may be generated by a fluorophore. A fluorophore may generate a signal upon release from a hybridization probe. A reaction may comprise excitation of a fluorophore. A reaction may comprise signal detection. A reaction may comprise detecting emission from a fluorophore.

A signal may be a fluorescent signal. A signal may correspond to a fluorescence intensity level. Each signal measured in the methods of the present disclosure may have a distinct fluorescence intensity value, thereby corresponding to the presence of a unique combination of nucleic acid targets. A signal may be generated by one or more oligonucleotide probes. Multiple signals may be generated by an oligonucleotide probe. For example, a oligonucleotide may be able to bind to multiple analytes and may generate a signal corresponding to hybridization with a first analyte and a second signal corresponding with a second analyte.

N may be a number of signals detected in a single optical channel in an assay of the present disclosure. N may be at least 2, 3, 4, 5, 6, 7, 8, 9, 10, 11, 12, 13, 14, 15, 20, 25, 30, 40, 50 or more. N may be at most 50, 40, 30, 24, 20, 15, 14, 13, 12, 11, 10, 9, 8, 7, 6, 5, 4, 3, or 2. N may be 2, 3, 4, 5, 6, 7, 8, 9, 10, 11, 12, 13, 14, 15, 20, 25, 30, 40, or 50.

As will be recognized and is described elsewhere herein, sets of signals may be generated in multiple different optical channels, where each set of signals is detected in a single optical channel, thereby significantly increasing the number of nucleic acid targets that can be measured in a single reaction. In some cases, two sets of signals are detected in a single reaction. Each set of signals detected in a reaction may comprise the same number of signals, or different numbers of signals.

In some cases, a signal may be generated simultaneous with hybridization of an oligonucleotide probe to a region of a nucleic acid. For example, an oligonucleotide probe (e.g, a molecular beacon probe or molecular torch) may generate a signal (e.g., a fluorescent signal) following hybridization to a nucleic acid. In some cases, a signal may be generated subsequent to hybridization of an oligonucleotide probe to a region of a nucleic acid, following degradation of the oligonucleotide probe by a nucleic acid enzyme.

In cases where an oligonucleotide probe comprises a signal tag, the oligonucleotide probe may be degraded when bound to a region of an oligonucleotide primer, thereby generating a signal. For example, an oligonucleotide probe (e.g., a TaqMan® probe) may generate a signal following hybridization of the oligonucleotide probe to a nucleic acid and subsequent degradation by a polymerase (e.g., during amplification, such as PCR amplification). An oligonucleotide probe may be degraded by the exonuclease activity of a nucleic acid enzyme.

An oligonucleotide probe may comprise a quencher and a fluorophore, such that the quencher is released upon degradation of an oligonucleotide probe, thereby generating a fluorescent signal. Thermal cycling may be used to generate one or more signals. Thermal cycling may generate at least 1, 2, 3, 4, 5, 6, 7, 8, 9, 10 signals, or more. Thermal cycling may generate at most 10, 9, 8, 7, 6, 5, 4, 3, 2, or 1 signal. Multiple signals may be of the same type or of different types. Signals of different types may be fluorescent signals with different fluorescent wavelengths. Signals of different types may be generated by detectable labels comprising different fluorophores. Signals of the same type may be of different intensities (e.g., different intensities of the same fluorescent wavelength). Signals of the same type may be signals detectable in the same color channel. Signals of the same type may be generated by detectable labels comprising the same fluorophore. Detectable labels comprising the same fluorophore may generate different signals by nature of being at different concentrations, thereby generating different intensities of the same signal type.

Although fluorescent probes have been used to illustrate this principle, the disclosed methods are equally applicable to any other method providing a quantifiable signal, including an electrochemical signal, chemiluminescent signals, magnetic particles, and electrets structures exhibiting a permanent dipole.

In certain portions of this disclosure, the signal may be a fluorescent signal. For example, like fluorescent signals, any of the electromagnetic signals described above may also be characterized in terms of a wavelength, whereby the wavelength of a fluorescent signal may also be described in terms of color. The color may be determined based on measuring intensity at a particular wavelength or range of wavelengths, for example by determining a distribution of fluorescent intensity at different wavelengths and/or by utilizing a band pass filter to determine the fluorescence intensity within a particular range of wavelengths.

The presence or absence of one or more signals may be detected. One signal may be detected, or multiple signals may be detected. Multiple signals may be detected simultaneously. Alternatively, multiple signals may be detected sequentially. A signal may be detected throughout the process of thermal cycling, for example, at the end of each thermal cycle. The signals may be detected in a multi-channel detector. For example, the signal may be observed using a detector that can observe a signal in multiple ranges of wavelengths simultaneously, substantially simultaneously, or sequentially. The signal may be observable in at least 1, 2, 3, 4, 5, 6, 7, 8, 9, 10, or more channels. The signal may be observable in no more than 10, 9, 8, 7, 6, 5, 4, 3, 2, or less channels.

In some cases, the signal intensity increases with each thermal cycle. The signal intensity may increase in a sigmoidal fashion. The presence of a signal may be correlated to the presence of at least one member of a plurality of target nucleic acids. Correlating the presence of a signal to the presence of at least one member of a plurality of target nucleic acids may comprise establishing a signal intensity threshold. A signal intensity threshold may be different for different signals. Correlating the presence of a signal to the presence of at least one member of a plurality of target nucleic acids may comprise determining whether the intensity of a signal increases beyond a signal intensity threshold. In some examples, the presence of a signal may be correlated with the presence of at least one of all members of a plurality of target nucleic acids. In other examples, the presence of a first signal may be correlated with the presence of at least one of a first subset of members of a plurality of target nucleic acids, and the presence of a second signal may be correlated with the presence of at least one of a second subset of members of a plurality of target nucleic acids.

The presence of a signal may be correlated to the presence of a nucleic acid target. The presence of least 1, 2, 3, 4, 5, 6, 7, 8, 9, 10, or more signals may be correlated with the presence of at least one of at least 1, 2, 3, 4, 5, 6, 7, 8, 9, 10, or more nucleic acid targets. The absence of a signal may be correlated with the absence of corresponding nucleic acid targets. The absence of least 1, 2, 3, 4, 5, 6, 7, 8, 9, 10, or more signals may be correlated with the absence of each of at least 1, 2, 3, 4, 5, 6, 7, 8, 9, 10, or more nucleic acid target molecules. The presence of a plurality of signals may be correlated with a combination of targets. The presence of a plurality of signals may be correlated with a unique combination of targets. For example, the detection of a particular plurality of signals may indicate the presence or absence of a unique or particular combination of targets.

### Accuracy

The method of the disclosure may be able to correctly identify the presence of analyte in a sample and can be measured in terms of the accuracy of the assay, the sensitivity of the assay, the specificity of the assay. Accuracy may be calculated as the total number of correctly classified samples divided by the total number of samples, e.g., in a test population. Sensitivity is a measure of the “true positives” that are predicted by a test to be positive, and may be calculated as the number of correctly identified cancer samples divided by the total number of cancer samples. Specificity is a measure of the “true negatives” that are predicted by a test to be negative, and may be calculated as the number of correctly identified normal samples divided by the total number of normal samples.

In some embodiments, the methods herein show an accuracy of at least about 75%, e.g., an accuracy of at least about 75%, about 80%, about 85%, about 90%, about 95%, about 97%, about 99% or about 100%. For example, the methods disclosed may predict the presence of an analyte at an accuracy of at least about 50%, at least about 55%, at least about 60%, at least about 65%, at least about 70%, at least about 75%, at least about 80%, at least about 85%, at least about 90%, at least about 95%, or more. In other embodiments, the methods herein show a specificity of at least about 75%, e.g., a specificity of at least about 75%, about 80%, about 85%, about 90%, about 95%, about 97%, about 99% or about 100%. For example, the method may identify the presence of an analytes at a specificity of at least about 50%, at least about 55%, at least about 60%, at least about 65%, at least about 70%, at least about 75%, at least about 80%, at least about 85%, at least about 90%, at least about 95%, or more. For example, the methods herein may identify the presence of an analyte a sensitivity of at least about 75%, e.g., a sensitivity of at least about 75%, about 80%, about 85%, about 90%, about 95%, about 97%, about 99% or about 100%. For example, the method may identify the presence the of an sensitivity of at least about 50%, at least about 55%, at least about 60%, at least about 65%, at least about 70%, at least about 75%, at least about 80%, at least about 85%, at least about 90%, at least about 95%, or more.

### Kits

The present disclosure provides kits for sample collection. The kit may comprise a sample collection vessel or sample collection tube. The kit may comprise a sample collection tool or an object that can obtain a sample via the contact of cells or nucleic acids from the subject and transfer sample to a sample collection vessel or tube. The sample collection tool may comprise a swab.

The present disclosure also provides kits for performing assays or analysis of assay results. Kits may comprise one or more oligonucleotide probes. Oligonucleotide probes may be lyophilized. Different oligonucleotide probes may be present at different concentrations in a kit. Oligonucleotide probes may comprise a fluorophore and/or one or more quenchers.

Kits may comprise one or more sets of oligonucleotide primers or oligonucleotide probes as described herein. The kits may further comprise a set of oligonucleotide primers comprising paired oligonucleotide primers. Paired oligonucleotide primers may comprise a forward oligonucleotide primer and a reverse oligonucleotide primer. A set of oligonucleotide primers may be configured to amplify a nucleic acid sequence corresponding to particular targets. For example, a forward oligonucleotide primer may be configured to hybridize to a first region (e.g., a 3′ end) of a nucleic acid sequence, and a reverse oligonucleotide primer may be configured to hybridize to a second region (e.g., a 5′ end) of the nucleic acid sequence, thereby being configured to amplify the nucleic acid sequence. Different sets of oligonucleotide primers may be configured to amplify nucleic acid sequences. In one example, a first set of oligonucleotide primers may be configured to amplify a first nucleic acid sequence, and a second set of oligonucleotide primers may be configured to amplify a second nucleic acid sequence. Oligonucleotide primers configured to amplify nucleic acid molecules may be used in performing the disclosed methods. In some cases, all of the oligonucleotide primers in a kit are lyophilized.

Kits may comprise one or more nucleic acid enzymes. A nucleic acid enzyme may be a nucleic acid polymerase. A nucleic acid polymerase may be a deoxyribonucleic acid polymerase (DNase). A DNase may be a Taq polymerase or variant thereof. A nucleic acid enzyme may be a ribonucleic acid polymerase (RNase). An RNase may be an RNase III. An RNase III may be Dicer. The nucleic acid enzyme may be an endonuclease. An endonuclease may be an endonuclease I. An endonuclease I may be a T7 endonuclease I. A nucleic acid enzyme may be capable of degrading a nucleic acid comprising a non-natural nucleotide. A nucleic acid enzyme may be an endonuclease V such as, for example, an E. coli endonuclease V. A nucleic acid enzyme may be a polymerase (e.g., a DNA polymerase). A polymerase may be Taq polymerase or a variant thereof. A nucleic acid enzyme may be capable, under appropriate conditions, of degrading an oligonucleotide probe. A nucleic acid enzyme may be capable, under appropriate conditions, of releasing a quencher from an oligonucleotide probe. Kits may comprise instructions for using any of the foregoing in the methods described herein.

### Systems

Methods as disclosed herein may be performed using a variety of systems. The systems may be configured such the steps of the method may be performed. For example, the systems may comprise a detector for the detection of signals as described elsewhere herein. The system may comprise a processor configured to process, receive, plot, or otherwise represent the data obtained from the detector. The processor may be configured to process the data as described elsewhere herein. The processor may be configured to generate a report of the results obtained from the assay. The results of the assay may be uploaded into a remote server, or other computer systems as described elsewhere herein. The results may be uploaded and sent to a subject's medical provider or an institution monitoring the spread of a disease. The results may also be sent to the subject directly. The subject, medical provider, or other institution may be able to access the remote server such review or analyze the results. For example, the results may then be transmitted to another institution/or medical professional for monitoring or for providing recommendations for the subject. In additional to the data generated for the detection of targets, the data may be used to monitor a geographical location of the assay or subject, for example to allow monitoring of the transmission of a disease. These results can then be uploaded into a cloud database or other remote database for storage and transmission to or access by a variety or individuals and institutions which may use the results of the assay. The results may be obtained on a smart phone or other computer system as disclosed elsewhere herein which may display the results.

The present disclosure provides computer systems that are programmed to implement methods of the disclosure. The computer system can perform various aspects of the present disclosure. The computer system can be an electronic device of a user or a computer system that is remotely located with respect to the electronic device. The electronic device can be a mobile electronic device.

The computer system may include a central processing unit (CPU, also “processor” and “computer processor” herein), which can be a single core or multi core processor, or a plurality of processors for parallel processing. The computer system may include memory or memory location (e.g., random-access memory, read-only memory, flash memory), electronic storage unit (e.g., hard disk), communication interface (e.g., network adapter) for communicating with one or more other systems, and peripheral devices, such as cache, other memory, data storage and/or electronic display adapters. The memory, storage unit, interface and peripheral devices are in communication with the CPU through a communication bus (solid lines), such as a motherboard. The storage unit can be a data storage unit (or data repository) for storing data. The computer system can be operatively coupled to a computer network (“network”) with the aid of the communication interface. The network can be the Internet, an internet and/or extranet, or an intranet and/or extranet that is in communication with the Internet. The network in some cases is a telecommunication and/or data network. The network can include one or more computer servers, which can enable distributed computing, such as cloud computing. The network, in some cases with the aid of the computer system, can implement a peer-to-peer network, which may enable devices coupled to the computer system to behave as a client or a server.

The CPU can execute a sequence of machine-readable instructions, which can be embodied in a program or software. The instructions may be stored in a memory location, such as the memory. The instructions can be directed to the CPU, which can subsequently program or otherwise configure the CPU to implement methods of the present disclosure. Examples of operations performed by the CPU can include fetch, decode, execute, and writeback.

The CPU can be part of a circuit, such as an integrated circuit. One or more other components of the system can be included in the circuit. In some cases, the circuit is an application specific integrated circuit (ASIC).

The storage unit can store files, such as drivers, libraries and saved programs. The storage unit can store user data, e.g., user preferences and user programs, or raw data or processed results from the assays. The computer system in some cases can include one or more additional data storage units that are external to the computer system, such as located on a remote server that is in communication with the computer system through an intranet or the Internet.

The computer system can communicate with one or more remote computer systems through the network. For instance, the computer system can communicate with a remote computer system of a user (e.g., operator). Examples of remote computer systems include personal computers (e.g., portable PC), slate or tablet PC's (e.g., Apple® iPad, Samsung® Galaxy Tab), telephones, Smart phones (e.g., Apple® iphone, Android-enabled device, Blackberry®), or personal digital assistants. The user can access the computer system via the network.

Methods as described herein can be implemented by way of machine (e.g., computer processor) executable code stored on an electronic storage location of the computer system, such as, for example, on the memory or electronic storage unit. The machine executable or machine-readable code can be provided in the form of software. During use, the code can be executed by the processor. In some cases, the code can be retrieved from the storage unit and stored on the memory for ready access by the processor. In some situations, the electronic storage unit can be precluded, and machine-executable instructions are stored on memory.

The code can be pre-compiled and configured for use with a machine having a processor adapted to execute the code, or can be compiled during runtime. The code can be supplied in a programming language that can be selected to enable the code to execute in a pre-compiled or as-compiled fashion.

Aspects of the systems and methods provided herein, such as the computer system, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system can include or be in communication with an electronic display that comprises a user interface (UI) for providing, for example, plots of data, plots of kinetic signatures, information relating to signal amplitude, Examples of UIs include, without limitation, a graphical user interface (GUI) and web-based user interface.

Methods and systems of the present disclosure can be implemented by way of one or more algorithms. An algorithm can be implemented by way of software upon execution by the central processing unit. The algorithm can, for example, parameterize data points or fit data point to specified mathematical functions, in order to quantify analytes.

### LIST OF EMBODIMENTS

Embodiment 1. A method of identifying the presence of an analyte in a sample, the method comprising:
   a) providing to said sample a primer oligonucleotide comprising a first region, wherein the first region hybridizes to said analyte and a second region comprising two or more distinct probe binding sites;
   b) subjecting said primer oligonucleotide to an extension reaction, thereby generating a probe binding nucleic acid;
   c) annealing (i) a second primer oligonucleotide and (ii) one or more probes to said probe binding nucleic acid;
   d) subjecting said second primer oligonucleotide to an extension reaction thereby generating one or more signals; and
   e) identifying the presence of said analyte based at least on detection of an intensity level and a wavelength of said one or more signals.
Embodiment 2. The method of embodiment 1, wherein said one or more signals is detected in more than one channel.
Embodiment 3. The method of any one of embodiments 1 or 2, wherein said one or more signals is two or more signals.
Embodiment 4. The method of any one of embodiments 1 to 3, wherein said one or more signals is three or more signals.
Embodiment 5. The method of any one of embodiments 1 to 4, wherein said one or more signals is four or more signals.
Embodiment 6. The method of any one of embodiments 1 to 5, wherein said one or more signals is detected in more than two channels.
Embodiment 7. The method of any one of embodiments 1 to 6, wherein said one or more signals is detected in more than three channels.
Embodiment 8. The method of any one of embodiments 1 to 7, wherein a signal of said one or more signals is detected at an intensity level of 1i.
Embodiment 9. The method of any one of embodiments 1 to 8, wherein a signal of said one or more signals is detected at an intensity level of 2i.
Embodiment 10. The method of any one of embodiments 1 to 9, wherein said one or more signals is generated from more than one probe that binds to said probe binding nucleic acid.
Embodiment 11. The method of any one of embodiments 1 to 10, wherein said one or more signals is generated via degradation of one or more probes that binds to said probe binding nucleic acid.
Embodiment 12. The method of any one of embodiments 1 to 11, wherein a probe of said one or more probes comprises fluorophore.
Embodiment 13. The method of embodiment 12, wherein said probe comprises a quencher.
Embodiment 14. The method of embodiment 12, wherein said fluorophore is selected from a group consisting of FAM, HEX, TAMRA, Cy5, and combinations thereof.
Embodiment 15. The method of any one of embodiments 1 to 14, wherein said second region comprises three or more distinct probe binding sites.
Embodiment 16. The method of any one of embodiments 1 to 15, wherein said second region comprises four distinct probe binding sites.
Embodiment 17. The method of any one of embodiments 1 to 16, prior to c), providing a first probe that binds to first probe binding site of said probe binding nucleic acid and a second probe that binds to a second probe binding site of said probe binding nucleic acid.
Embodiment 18. The method of embodiment 17, wherein said first probe is present in a same concentration as a concentration of said second probe.
Embodiment 19. The method of embodiment 17, wherein said first probe is present in a different concentration as a concentration of said second probe.
Embodiment 20. The method of embodiment 17, wherein said first probe is present in a greater concentration than a concentration of said second probe.
Embodiment 21. The method of embodiment 17, wherein said first probe is present in more than two times greater concentration than a concentration of said second probes.
Embodiment 22. The method of any one of embodiments 1 to 21, wherein a) further comprises providing a first reverse oligonucleotide that hybridizes to said analyte.
Embodiment 23. The method of any one of embodiments 1 to 22, wherein b) comprises performing an amplification reaction to generate a plurality of amplicons of said probe binding nucleic acid.
Embodiment 24. The method of any one of embodiments 1 to 23, further comprising, subsequent to a) and prior to b) generating a plurality of partitions.
Embodiment 25. The method of any one of embodiments 1 to 24, wherein said analyte comprises a nucleic acid.
Embodiment 26. The method of embodiment 25, wherein said nucleic acid comprises a DNA or RNA.
Embodiment 27. The method of any one of embodiments 1 to 26, wherein said first primer oligonucleotide further comprises a third region 5′ to said second and first region.
Embodiment 28. The method of embodiment 27, wherein c) comprises annealing said second primer oligonucleotide to said third region.
Embodiment 29. The method of any one of embodiments 1 to 28, wherein c) comprises annealing said second primer oligonucleotide to at least a portion of said second region.
Embodiment 30. The method of any one of embodiments 1 to 29, further comprising, identifying the presence of a second analyte, wherein a) comprises providing a third primer oligonucleotide comprising a first region that hybridizes to said second analyte and a second region comprising one or more probe binding sites.
Embodiment 31. The method of any of embodiments 1 to 30, wherein the analyte is detected at a sensitivity of a least 95%.
Embodiment 32. The method of any of embodiments 1 to 31, wherein the analyte is detected at a sensitivity of a least 99%.
Embodiment 33. A method of identifying the presence of one or more analytes of a plurality of analytes in a sample, the method comprising:
   a) providing to said sample a plurality of tailed primers wherein a first tailed primer of said plurality of tailed primers comprises a first region that hybridizes to an analyte and a second region comprising a first set of one or more probe binding sites, and a second tailed primer of said plurality of tailed primers comprises a first region that hybridizes to a second analyte and a second region comprising a second set of one or more probe binding sites, wherein said first set of probe binding sites and said second set of probe binding sites are not identical;
   b) subjecting said plurality of tailed primers to extension reactions, thereby generating probe binding nucleic acids;
   c) annealing a plurality of second primers to said probe binding nucleic acids;
   d) subjecting said plurality of second primers to extension reactions to generate (i) if the first analyte is present, a first set of one or more signals, and (ii) if the second analyte is present, a second set of one or more signals; and
   e) based at least on if present, said first set of one or more signals and, if present, said second set of one or more signals, identifying the presence of one or more analytes of the plurality of analytes.
Embodiment 34. The method of embodiment 33, wherein d) comprises generating (i) if said first analyte is present, a first set of one or more signals from one or more probes that bind said first set of probe binding sites, and (ii) if said second analyte is present, a second set of one or more signals from one or more probes that bind said second set of probe binding sites.
Embodiment 35. The method of any one of embodiments 33 or 34, wherein said first set of one or more signals or second set of one or more signals is detected in more than one channel.
Embodiment 36. The method of any one of embodiments 33 to 35, wherein said first set of one or more signals or second set of one or more signals is detected in more than two channels.
Embodiment 37. The method of any one of embodiments 33 to 36, wherein said first set of one or more signals or second set of one or more signals is detected in more than three channels.
Embodiment 38. The method of any one of embodiments 33 to 37, wherein said first set of one or more signals or second set of one or more signals is detected in more than four channels.
Embodiment 39. The method of any one of embodiments 33 to 38, wherein said identifying the presence of the plurality of analytes is based at least on identifying an intensity level of the signals of the first or second set of one or more signals.
Embodiment 40. The method of any one of embodiments 33 to 39, wherein a signal of said first or second set of one or more signals is detected at an intensity level of 1i.
Embodiment 41. The method of any one of embodiments 33 to 40, wherein a signal of said first or second set of one or more signals is detected at an intensity level of 2i.
Embodiment 42. The method of any one of embodiments 33 to 41, wherein a first signal of the first set of one or more signals is detected in a first channel at an intensity of 1i and a second signal of the first set of one or more signals is detecting in a second channel at an intensity of 1i.
Embodiment 43. The method of any one of embodiments 33 to 42, wherein a first signal of the first set of one or more signals is detected in a first channel at an intensity of 1i and a second signal of the first set of one or more signals is detecting in a second channel at an intensity of 2i.
Embodiment 44. The method of any one of embodiment 34 to 43, wherein said first or second set of one or more signals is generated from more than one probe that binds to said probe binding nucleic acid.
Embodiment 45. The method of any one of embodiment 34 to 44, wherein the first or second set of one or more signals is generated via degradation of said a probe that binds to said probe binding nucleic acid.
Embodiment 46. The method of any one of embodiment 33 to 45, further comprising, subsequent to b) and prior to d), annealing one or more probes to the first or second set of probe binding sites.
Embodiment 47. The method of embodiment 46, wherein a probe of said one or more probes comprises fluorophore.
Embodiment 48. The method of embodiment 47, wherein said probe comprises a quencher.
Embodiment 49. The method of embodiment 47, wherein said fluorophore is selected from a group consisting of FAM, HEX, TAMRA, Cy5, and combinations thereof.
Embodiment 50. The method of any one of embodiment 33 to 49, wherein said second region of the first tailed primer or the second tailed primer comprises two or more probe binding sites.
Embodiment 51. The method of any one of embodiment 33 to 50, wherein said second region of the first tailed primer or the second tailed primer comprises three or more probe binding sites.
Embodiment 52. The method of any one of embodiment 33 to 51, wherein said second region of the first tailed primer or the second tailed primer comprises four probe binding sites.
Embodiment 53. The method of any one of embodiment 33 to 52, prior to d), providing a first probe that binds to first probe binding site of said probe binding nucleic acids and a second probe that binds to a second probe binding site of said probe binding nucleic acids.
Embodiment 54. The method of embodiment 53, wherein said first probe is present in a same concentration as a concentration of said second probe.
Embodiment 55. The method of embodiment 53, wherein said first probe is present in a different concentration as a concentration of said second probe.
Embodiment 56. The method of embodiment 53, wherein said first probe is present in a greater concentration than a concentration of said second probe.
Embodiment 57. The method of embodiment 53, wherein said first probe is present in more than 2 times greater concentration than a concentration of said second probes.
Embodiment 58. The method of any one of embodiment 33 to 57, wherein a) further comprises providing a third oligonucleotide comprising a region substantially complementary to said analyte.
Embodiment 59. The method of any one of embodiment 33 to 58, wherein b) comprises performing an amplification reaction to generate a plurality of amplicons of said probe binding nucleic acids.
Embodiment 60. The method any one of embodiment 33 to 58, further comprising, subsequent to a) and prior to b) generating a plurality of partitions.
Embodiment 61. The method of embodiment 60, wherein a first partition of said plurality of partition comprises said first analyte and a second partition of said plurality of partition comprises said second analytes.
Embodiment 62. The method of embodiment 61, wherein said first set of one or more signals are generated in said first partition and said second set of signal and generated in said second partition.
Embodiment 63. The method of embodiment 62, further comprising, detecting said first set of one or more signals separately from detecting said second set of one or more signals.
Embodiment 64. The method of any of embodiments 33 to 63, wherein said plurality of analytes comprises a nucleic acid.
Embodiment 65. The method of embodiment 64, wherein said nucleic acid comprises a DNA or RNA.
Embodiment 66. The method of any of embodiments 33 to 65, wherein said first tailed primer and said second tailed primer each further comprise a third region 5′ to said second and first region.
Embodiment 67. The method of embodiment 66, wherein c) comprises annealing said plurality of second primers to said third region.
Embodiment 68. The method of any of embodiments 33 to 67, wherein c) comprises annealing second primers of the plurality of second primers to at least a portion of said second region of said first tailed primer or said second tailed primer.
Embodiment 69. The method of any of embodiments 33 to 68, wherein the first set of probe binding sites and said the second set of probe binding sites comprise a same sequence.
Embodiment 70. The method of any of embodiments 33 to 69, wherein the first set of probe binding sites and said the second set of probe binding sites comprise a same probe binding sequence.
Embodiment 71. The method of any of embodiment 33 to 69, wherein the one or more analytes comprises two or more analytes.
Embodiment 72. The method of any of embodiments 33 to 71, wherein the two or more analytes comprises a first analyte present at a concentration of no more than 5% of a concentration of a second analyte.
Embodiment 73. The method of any of embodiments 33 to 72, wherein the two or more analytes comprises a first analyte present at a concentration of no more than 2% of a concentration of a second analyte.
Embodiment 74. The method of any of embodiments 33 to 73, wherein the two or more analytes comprises a first analyte present at a concentration of no more than 1% of a concentration of a second analyte.
Embodiment 75. The method of any of embodiments 33 to 74, wherein the two or more analytes comprises a first analyte present at a concentration of no more than 0.1% of a concentration of a second analyte.
Embodiment 76. The method of any of embodiments 33 to 75, wherein the one or more analytes are detected at a sensitivity of a least 95%.
Embodiment 77. The method of any of embodiments 33 to 76, wherein the one or more analytes are detected at a sensitivity of a least 99%.
Embodiment 78. An oligonucleotide primer comprising a first region substantially complementary to a nucleic acid target and a second region comprising two or more probe binding sites, wherein one or more of the two or more probe binding site is configured to anneal to an oligonucleotide probe that generates a detectable signal upon either of: (i) binding to said probe binding sites or (ii) degradation of said oligonucleotide probe.
Embodiment 79. The oligonucleotide primer of embodiment 78, further comprising a third region 5′ to said first region and said second region.
Embodiment 80. The oligonucleotide primer of any of embodiments 78 or 79, wherein said first region is 3′ to said second region.
Embodiment 81. The oligonucleotide primer of any of embodiments 78 or 80, wherein said two or more probe binding sites comprises three or more probe binding sites.
Embodiment 82. The oligonucleotide primer of any of embodiments 78 or 81, wherein said two or more probe binding sites comprises four probe binding sites.
Embodiment 83. A composition comprising:
   (i) an oligonucleotide comprising a first region substantially complementary to a first region of a nucleic acid target and a second region comprising two or more probe binding sites; and
   (ii) a plurality of probes, wherein a probe of said plurality of probes is configured to anneal to said one or more probe binding sites, or complements thereof.
Embodiment 84. The composition of embodiment 83, wherein said oligonucleotide further comprises a universal primer binding region 5′ to said first region and said second region.
Embodiment 85. The composition of embodiment 84, further comprising a universal primer oligonucleotide comprising a region substantially complementary or homologous to said universal primer binding.
Embodiment 86. The composition of any of embodiments 83 to 85, further comprising a primer binding oligonucleotide comprising a region substantially complementary or homologous to a sequence of said oligonucleotide.
Embodiment 87. The composition of any of embodiments 83 to 86, wherein said first region is 3′ to said second region.
Embodiment 88. The composition of any of embodiments 83 to 87, wherein the oligonucleotide further comprises a third region 5′ to said first region and said second region, wherein said third region is substantially complementary to said nucleic acid target at a sequence different than said first region of said nucleic acid target.
Embodiment 89. The composition of any of embodiments 83 to 88, wherein said two or more probe binding sites comprises three or more probe binding sites.
Embodiment 90. The composition of any of embodiments 83 to 89, wherein said two or more probe binding sites comprises four probe binding sites.
Embodiment 91. The composition of any of embodiments 83 to 90, wherein a first probe of the plurality of probes is present in a same concentration as a concentration of a second probe of the plurality of probes.
Embodiment 92. The composition of any of embodiments 83 to 91, wherein a first probe of the plurality of probes is present in a different concentration as a concentration of a second probe of the plurality of probes.
Embodiment 93. The composition of any of embodiments 83 to 92, wherein the concentration of a first probe of the plurality of probes is greater the two times the concentration of a second probe of the plurality of probes.
Embodiment 94. The composition of any of embodiments 83 to 93, further comprising a reverse oligonucleotide comprising a region substantially complementary to a second region of said nucleic acid target.
Embodiment 95. The composition of any of embodiments 83 to 94, further comprising a second oligonucleotide comprising a first region substantially complementary to a first region of a second nucleic acid target and a second region comprising one or more probe binding sites.
Embodiment 96. The composition of any of embodiments 83 to 95, further comprising a tail oligonucleotide comprising a first region substantially complementary to a first region of a second nucleic acid target and a second region comprising one or more probe binding sites.
Embodiment 97. A composition comprising:
   (i) a plurality of oligonucleotides, wherein each oligonucleotide of said plurality of oligonucleotides comprises a first region substantially complementary to a nucleic acid target and a second region comprising a set of one or more probe binding sites, wherein the set of one or more probe binding sites for a first oligonucleotide is different than the set of one or more probe binding sites for a second oligonucleotide, wherein said set of one or more probe binding sites for said first oligonucleotide and said set of one or more probe binding sites for a second oligonucleotide comprise a probe binding site that is the same; and
   (ii) a plurality of probes, wherein a probe of said plurality of probes is configured to anneal to said one or more probe binding sites, or complements thereof.
Embodiment 98. The composition of embodiment 97, wherein each oligonucleotide further comprises a universal primer binding 5′ to said first region and said second region.
Embodiment 99. The composition of embodiment 98, further comprising a universal primer oligonucleotide comprising a region substantially complementary or homologous to said universal primer binding region.
Embodiment 100. The composition of any of embodiments 97 to 99, further comprising a primer binding oligonucleotide comprising a region substantially complementary or homologous to a sequence of said plurality of oligonucleotides.
Embodiment 101. The composition of any of embodiments 97 to 100, wherein said first region is 3′ to said second region.
Embodiment 102. The composition of any of embodiments 97 to 101, wherein the oligonucleotide further comprises a third region 5′ to said first region and said second region, wherein said third region is substantially complementary to said nucleic acid target at a sequence different than said first region of said nucleic acid target.
Embodiment 103. The composition of any of embodiments 97 to 102, wherein said one or more probe binding sites comprises two or more probe binding sites.
Embodiment 104. The composition of any of embodiments 97 to 103, wherein said one or more probe binding sites comprises three or more probe binding sites.
Embodiment 105. The composition of any of embodiments 97 to 104, wherein said one or more probe binding sites comprises four probe binding sites.
Embodiment 106. The composition of any of embodiments 97 to 105, wherein a first probe of the plurality of probes is present in a same concentration as a concentration of a second probe of the plurality of probes.
Embodiment 107. The composition of any of embodiments 97 to 106, wherein a first probe of the plurality of probes is present in a different concentration as a concentration of a second probe of the plurality of probes.
Embodiment 108. The composition of any of embodiments 97 to 107, wherein the concentration of a first probe of the plurality of probes is greater the two times the concentration of a second probe of the plurality of probes.
Embodiment 109. The composition any of embodiments 97 to 108, further comprising a reverse oligonucleotide comprising a region substantially complementary to a second region of said nucleic acid target.
Embodiment 110. The composition any of embodiments 97 to 109, further comprising a secondary oligonucleotide comprising a first region substantially complementary to a first region of a second nucleic acid target and a second region comprising one or more probe binding sites.
Embodiment 111. A composition comprising: (i) a plurality of probe oligonucleotides, wherein at least two probe oligonucleotides of said plurality of probe oligonucleotides comprise a same nucleic acid sequence and a fluorophore and wherein each fluorophore of said at least two probe oligonucleotide is different.
Embodiment 112. The composition of embodiment 111, further comprising a first primer configured to anneal to a first region of an analyte, and a second primer configured to anneal to a second region of an analyte.
Embodiment 113. The composition of embodiment 112, further comprising a third primer configured to anneal to a first region of a second analyte, and a fourth primer configured to anneal to a second region of second analyte.
Embodiment 114. The composition of any of embodiments 111 to 113, wherein at least three probe oligonucleotides of said plurality of probes comprise a same nucleic acid sequence and a fluorophore and wherein each fluorophore of said at least three probe oligonucleotide is different.
Embodiment 115. The composition of any of embodiments 111 to 114, wherein at least four probe oligonucleotides of said plurality of probes comprise a same nucleic acid sequence and a fluorophore and wherein each fluorophore of said at least four probe oligonucleotide is different.
Embodiment 116. The composition of any of embodiments 111 to 115, wherein each probe of said plurality of probe oligonucleotides is at a same concentration as other probes comprising different sequences.
Embodiment 117. A method of identifying the presence of an analyte in a sample, the method comprising:
   a) providing to said sample an oligonucleotide comprising, from 5′ to 3′, a first region that hybridizes to said analyte at a first sequence, a second region comprising two or more distinct probe binding sites, and a third region that hybridizes to said analyte at a second sequence adjacent to said first sequence;
   b) subjecting said oligonucleotide to a circularization reaction, thereby generating a circularized probe binding nucleic acid;
   d) annealing a (i) primer oligonucleotide and (ii) one or more probes to said circularized probe binding nucleic acid, or derivatives thereof;
   e) subjecting said primer oligonucleotide to an extension reaction, thereby generating one or more signals; and
   f) identifying the presence of said analyte based at least on detection of an intensity level and wavelength of said one or more signals.
Embodiment 118. The method of embodiment 117, wherein prior to (b), said oligonucleotide hybridizes to said analyte at said first sequence and said second sequence.
Embodiment 119. The method of any of embodiments 117 or 118, further comprising, subsequent to (b) and prior to (d), cleaving said circularized probe binding nucleic acids to generate a linearized probe binding nucleic acid, wherein (d) comprises annealing said primer oligonucleotide and said one or more probes to said linearized probe binding nucleic acids.
Embodiment 120. The method of any of embodiments 117 to 119, further comprising subsequent to (b), and prior to (d), subjecting said sample to an exonuclease reaction.
Embodiment 121. The method of embodiment 120, wherein said exonuclease reaction degrades oligonucleotides that fail to anneal to an analyte.
Embodiment 122. The method of any of embodiments 117 to 121, wherein said one or more signals is detected in more than one channel.
Embodiment 123. The method of any of embodiments 117 to 122, wherein said one or more signals is two or more signals.
Embodiment 124. The method of any of embodiments 117 to 123, wherein said one or more signals is three or more signals.
Embodiment 125. The method of any of embodiments 117 to 124, wherein said one or more signals is four or more signals.
Embodiment 126. The method of any of embodiments 117 to 125, wherein said one or more signals is detected in more than two channels.
Embodiment 127. The method of any of embodiments 117 to 126, wherein said one or more signals is detected in more than three channels.
Embodiment 128. The method of any of embodiments 117 to 127, wherein a signal of said one or more signals is detected at an intensity level of 1i.
Embodiment 129. The method of any of embodiments 117 to 128, wherein a signal of said one or more signals is detected at an intensity level of 2i.
Embodiment 130. The method of any of embodiments 117 to 129, wherein said one or more signals is generated from more than one probe that binds to said probe binding nucleic acid.
Embodiment 131. The method of any of embodiments 117 to 130, wherein said one or more signals is generated via degradation of one or more probes that binds to said probe binding nucleic acid.
Embodiment 132. The method of any of embodiments 117 to 131, wherein a probe of said one or more probes comprises fluorophore.
Embodiment 133. The method of embodiment 132, wherein said probe comprises a quencher.
Embodiment 134. The method of embodiment 132, wherein said fluorophore is selected from a group consisting of FAM, HEX, TAMRA, Cy5, and combinations thereof.
Embodiment 135. The method of any of embodiments 117 to 136, wherein said second region comprises three or more distinct probe binding sites.
Embodiment 136. The method of any of embodiments 117 to 137, wherein said second region comprises four distinct probe binding sites.
Embodiment 137. The method of any of embodiments 117 to 138, prior to d), providing a first probe that binds to first probe binding site of said probe binding nucleic acid and a second probe that binds to a second probe binding site of said probe binding nucleic acid.
Embodiment 138. The method of embodiment 137, wherein said first probe is present in a same concentration as a concentration of said second probe.
Embodiment 139. The method of embodiment 137, wherein said first probe is present in a different concentration as a concentration of said second probe.
Embodiment 140. The method of embodiment 137, wherein said first probe is present in a greater concentration than a concentration of said second probe.
Embodiment 141. The method of embodiment 137, wherein said first probe is present in more than two times greater concentration than a concentration of said second probes.
Embodiment 142. The method of any of embodiments 117 to 141, wherein e) further comprises providing a reverse primer that hybridizes to said linearized probe binding nucleic acid.
Embodiment 143. The method of any of embodiments 117 to 142, wherein e) comprises performing an amplification reaction to generate a plurality of amplicons of said linearized probe binding nucleic acid.
Embodiment 144. The method of any of embodiments 117 to 143, further comprising, subsequent to a) and prior to b) generating a plurality of partitions.
Embodiment 145. The method of any of embodiments 117 to 144, wherein said analyte comprises a nucleic acid.
Embodiment 146. The method of embodiment 145, wherein said nucleic acid comprises a DNA or RNA.
Embodiment 147. The method of any of embodiments 117 to 146, wherein said first primer oligonucleotide further comprises a third region 5′ to said second and first region.
Embodiment 148. The method of embodiment 147, wherein d) comprises annealing said second primer oligonucleotide to said third region.
Embodiment 149. The method of any of embodiments 117 to 148, wherein d) comprises annealing said second primer oligonucleotide to at least a portion of said second region.
Embodiment 150. The method of any of embodiments 117 to 149, further comprising, identifying the presence of a second analyte, wherein a) comprises providing a third primer oligonucleotide comprising a first region that hybridizes to said second analyte and a second region comprising one or more probe binding sites.
Embodiment 151. The method of embodiment 150, wherein said third primer oligonucleotide comprises from 5′ to 3′, a first region that hybridizes to said second analyte at a first sequence, a second region comprising one or more probe binding sites, and a third region configured to hybridize to said second analyte at a second sequence adjacent to said first sequence of said second analyte.
Embodiment 152. The method of any of embodiments 117 to 151, wherein the analyte is detected at a sensitivity of a least 95%.
Embodiment 153. The method of any of embodiments 117 to 152, wherein the analyte is detected at a sensitivity of a least 99%.
Embodiment 154. A method of identifying the presence of one or more analytes of a plurality of analytes in a sample, the method comprising:
   a) providing to said sample a plurality of molecular inversion oligonucleotides wherein a first molecular inversion oligonucleotide of said plurality of molecular inversion oligonucleotides comprises, from 5′ to 3′, a first region that hybridizes a first sequence of said analyte, a second region comprising a first set of one or more probe binding sites, and a third region that hybridizes to a second sequence of said analyte that is adjacent to said first sequence of said analyte, and a second molecular inversion oligonucleotide of said plurality of molecular inversion oligonucleotides comprises, from 5′ to 3′, a first region that hybridizes a first sequence of a second analyte, a second region comprising a second set of one or more probe binding sites, and a third region that hybridizes to a second sequence of said second analyte that is adjacent to said first sequence of said second analyte, wherein said first set of probe binding sites and said second set of probe binding sites are not identical;
   b) subjecting said plurality of molecular inversion oligonucleotides to circularization reactions, thereby generating circularized probe binding nucleic acids;
   c) cleaving said circularized probe binding nucleic acids to generate linearized probe binding nucleic acids;
   d) annealing a plurality of second primers to said linearized probe binding nucleic acids;
   e) subjecting said plurality of second primers to extension reactions to generate (i) if the first analyte is present, a first set of one or more signals, and (ii) if the second analyte is present, a second set of one or more signals; and
   f) based at least on if present, said first set of one or more signals and, if present, said second set of one or more signals, identifying the presence of one or more analytes of the plurality of analytes.
Embodiment 155. The method of embodiment 154, wherein e) comprises generating (i) if said first analyte is present, a first set of one or more signals from one or more probes that bind said first set of probe binding sites, and (ii) if said second analyte is present, a second set of one or more signals from one or more probes that bind said second set of probe binding sites.
Embodiment 156. The method of any of embodiments 154 or 155, wherein said first set of one or more signals or second set of one or more signals is detected in more than one channel.
Embodiment 157. The method of any of embodiments 154 to 156, wherein said first set of one or more signals or second set of one or more signals is detected in more than two channels.
Embodiment 158. The method of any of embodiments 154 to 157, wherein said first set of one or more signals or second set of one or more signals is detected in more than three channels.
Embodiment 159. The method of any of embodiments 154 to 158, wherein said first set of one or more signals or second set of one or more signals is detected in more than four channels.
Embodiment 160. The method of any of embodiments 154 to 159, wherein said identifying the presence of the plurality of analytes is based at least on identifying an intensity level of the signals of the first or second set of one or more signals.
Embodiment 161. The method of any of embodiments 154 to 160, wherein a signal of said first or second set of one or more signals is detected at an intensity level of 1i.
Embodiment 162. The method of any of embodiments 154 to 161, wherein a signal of said first or second set of one or more signals is detected at an intensity level of 2i.
Embodiment 163. The method of any of embodiments 154 to 162, wherein a first signal of the first set of one or more signals is detected in a first channel at an intensity of 1i and a second signal of the first set of one or more signals is detecting in a second channel at an intensity of 1i.
Embodiment 164. The method of any of embodiments 154 to 163, wherein a first signal of the first set of one or more signals is detected in a first channel at an intensity of 1i and a second signal of the first set of one or more signals is detecting in a second channel at an intensity of 2i.
Embodiment 165. The method of any of embodiments 155 to 164, wherein said first or second set of one or more signals is generated from more than one probe that binds to said probe binding nucleic acid.
Embodiment 166. The method of any of embodiments 155 to 165, wherein the first or second set of one or more signals is generated via degradation of said a probe that binds to said probe binding nucleic acid.
Embodiment 167. The method of any of embodiments 154 to 166, further comprising, subsequent to c) and prior to e), annealing one or more probes to the first or second set of probe binding sites.
Embodiment 168. The method of embodiment 167, wherein a probe of said one or more probes comprises fluorophore.
Embodiment 169. The method of embodiment 168, wherein said probe comprises a quencher.
Embodiment 170. The method of embodiment 168, wherein said fluorophore is selected from a group consisting of FAM, HEX, TAMRA, Cy5, and combinations thereof.
Embodiment 171. The method of any of embodiments 154 to 170, wherein said second region of the first molecular inversion oligonucleotide or the second molecular inversion oligonucleotide comprises two or more probe binding sites.
Embodiment 172. The method of any of embodiments 154 to 171, wherein said second region of the first molecular inversion oligonucleotide or the second molecular inversion oligonucleotide comprises three or more probe binding sites.
Embodiment 173. The method of any of embodiments 154 to 172, wherein said second region of the first molecular inversion oligonucleotide or the second molecular inversion oligonucleotide comprises four probe binding sites.
Embodiment 174. The method of any of embodiments 154 to 173, prior to d), providing a first probe that binds to first probe binding site of said probe binding nucleic acids and a second probe that binds to a second probe binding site of said probe binding nucleic acids.
Embodiment 175. The method of embodiment 174, wherein said first probe is present in a same concentration as a concentration of said second probe.
Embodiment 176. The method of embodiment 174, wherein said first probe is present in a different concentration as a concentration of said second probe.
Embodiment 177. The method of embodiment 174, wherein said first probe is present in a greater concentration than a concentration of said second probe.
Embodiment 178. The method of embodiment 174, wherein said first probe is present in more than 2 times greater concentration than a concentration of said second probes.
Embodiment 179. The method of any of embodiments 154 to 178, wherein d) further comprises providing a third primer oligonucleotide that hybridizes to said linearized probe binding nucleic acids.
Embodiment 180. The method of any of embodiments 154 to 179, wherein e) comprises performing an amplification reaction to generate a plurality of amplicons of said linearized probe binding nucleic acids.
Embodiment 181. The method of any of embodiments 154 to 180, further comprising, subsequent to a) and prior to b) generating a plurality of partitions.
Embodiment 182. The method of embodiment 181, wherein a first partition of said plurality of partition comprises said first analyte and a second partition of said plurality of partition comprises said second analytes.
Embodiment 183. The method of embodiment 182, wherein said first set of one or more signals are generated in said first partition and said second set of signal and generated in said second partition.
Embodiment 184. The method of embodiment 183, further comprising, detecting said first set of one or more signals separately from detecting said second set of one or more signals.
Embodiment 185. The method of any of embodiments 154 to 184, wherein said plurality of analytes comprises a nucleic acid.
Embodiment 186. The method of embodiment 185, wherein said nucleic acid comprises a DNA or RNA.
Embodiment 187. The method of any of embodiments 154 to 186, wherein said first molecular inversion oligonucleotide and said second molecular inversion oligonucleotide each further comprise a third region.
Embodiment 188. The method of embodiment 187, wherein c) comprises annealing said plurality of second primers to said third region.
Embodiment 189. The method of any of embodiments 154 to 188, wherein c) comprises annealing second primers of the plurality of second primers to at least a portion of said second region of said first molecular inversion oligonucleotide or said second molecular inversion oligonucleotide.
Embodiment 190. The method of any of embodiments 154 to 189, wherein the first set of probe binding sites and said the second set of probe binding sites comprise a same sequence.
Embodiment 191. The method of any of embodiments 154 to 190, wherein the first set of probe binding sites and said the second set of probe binding sites comprise a same probe binding sequence.
Embodiment 192. The method of any of embodiments 154 to 191, wherein the one or more analytes comprises two or more analytes.
Embodiment 193. The method of any of embodiments 154 to 192, wherein the two or more analytes comprises a first analyte present at a concentration of no more than 5% of a concentration of a second analyte.
Embodiment 194. The method of any of embodiments 154 to 193, wherein the two or more analytes comprises a first analyte present at a concentration of no more than 2% of a concentration of a second analyte.
Embodiment 195. The method of any of embodiments 154 to 194, wherein the two or more analytes comprises a first analyte present at a concentration of no more than 1% of a concentration of a second analyte.
Embodiment 196. The method of any of embodiments 154 to 195, wherein the two or more analytes comprises a first analyte present at a concentration of no more than 0.1% of a concentration of a second analyte.
Embodiment 197. The method of any of embodiments 154 to 196, wherein the one or more analytes are detected at a sensitivity of a least 95%.
Embodiment 198. The method of any of embodiments 154 to 197, wherein the one or more analytes are detected at a sensitivity of a least 99%.
Embodiment 199. An oligonucleotide comprising, from 5′ to 3′, a first region configured to hybridize to said analyte at a first sequence, a second region comprising two or more distinct probe binding sites, and a third region configured to hybridize to said analyte at a second sequence adjacent to said first sequence, wherein one or more of the two or more probe binding site is configured to anneal to an oligonucleotide probe that generates a detectable signal upon either of: (i) binding to said probe binding sites or (ii) degradation of said oligonucleotide probe.
Embodiment 200. The oligonucleotide primer of embodiment 199, wherein said two or more probe binding sites comprises three or more probe binding sites.
Embodiment 201. The oligonucleotide primer of embodiment 199 or 200, wherein said two or more probe binding sites comprises four probe binding sites.

### EXAMPLES

### Example 1: Detection of Multiple Analytes

An assay is set up to analyze multiple nucleic acid targets.

A 2X PCR Master Mix is thawed at room temperature. The reaction mix is prepared for a full plate with 7.2 μl of Reaction Mix added to strip tubes. 4.8 of Sample is added to strip tubes and the tubes are. capped strip tubes. The strips are vortexed for 10 seconds and spun down, and then vortexed and spun again 9 μl of Reaction Mix/Sample mixture is dispensed using reverse pipetting to the designated well, and 12 μl of Isolation buffer to each well. The plate is capped and then loaded on to an AbsoluteQ dPCR Instrument and ran using standard following Cycling parameters:

### Cycling Parameters

Initial Denature
   95° C. 10 minutes
3 Step Cycling 40×
   95° C. 10 seconds
   60° C. 30 seconds
   68° C. 60 seconds

### Array Scanning

Signals from the probes are detected via the Instrument and are analyzed based on intensity and channel. Based on the signals detected, a determination is made regarding which analytes are present.

Table 2 shows a set of example primers and probes that are used to analyze different samples.

TABLE 2
Example Primer and Probes

| | |
|---|---|
| BRAF_V600E_ASP1_ | GACCGACATCGCACGCTatATGATCGCCCGTA |
| 1xx0 | atATAACCGTCCGCTatCCACTCCATCGAGAT |
| | TTCTrCTGTAC/3SpC3/ |
| EGFR_L861Q_ASP2_ | GACCGACATCGCACGCTatACGCCGCTAACTA |
| 0xx1 | atACCGCCGACTTATatCTTCCGCACCCAGCT |
| | rGTTTGc/3SpC3/ |
| COSM20959_ASP2_ | GACCGACATCGCACGCTatATGATCGCCCGTA |
| 1xx1 | atACCGCCGACTTATatGAAGCATACGTGATG |
| | GCArUACGTC/3SpC3/ |
| BRAF_V600E_LSP1 | GCCTGTTTTCCTTTACTTACTACACCTrCAGA |
| | TT/3SpC3/ |
| EGFR_L861Q_LSP2 | GCACCGCAGCATGTCAAGATrCACAGt/ |
| | 3SpC3/ |
| COSM20959_LSP2 | GCTTTCCCGGACATGGTCTArAGAGGG/ |
| | 3SpC3/ |
| Universal | GACCGACATCGCACGCT |
| Forward Primer | |
| Universal Probe | A+T+GATCG+CC+CG+TA |
| Dye 1 | |
| Universal Probe | A+C+CG+C+CGACTT+AT |
| Dye 4 | |

Table 3 shows an example primer probe mix at 20×.

TABLE 3
Primer Probe Mix 20X

| | | Final |
|---|---|---|
| | 20X | Concentration |
| Barcoded Allele Specific | 1 uM | 50 nM |
| Primer Assay 1 | | |
| Locus Specific Primer Assay 1 | 6 uM | 300 nM |
| Barcoded Allele Specific | 1 uM | 50 nM |
| Primer Assay 2 | | |
| Locus Specific Primer Assay 2 | 6 uM | 300 nM |
| . | | |
| . | | |
| . | | |
| Barcoded Allele Specific | 1 uM | 50 nM |
| Primer Assay 15 | | |
| Locus Specific Primer Assay 15 | 6 uM | 300 nM |
| Universal Forward Primer | 6 uM | 300 nM |
| Universal Probe Dye 1 | 1 uM | 50 nM |
| Universal Probe Dye 2 | 1 uM | 50 nM |
| Universal Probe Dye 3 | 1 uM | 50 nM |
| Universal Probe Dye 4 | 1 uM | 50 nM |

Table 4 shows an example reaction mix

TABLE 4
Reaction Mix

| | 1 reaction | 18 reactions |
|---|---|---|
| 2X Master Mix | 6 μL | 108 μL |
| 20X Primer Probe Mix | 0.6 μL | 10.8 μL |
| 20X Passive Reference Dye 1 μM ROX | 0.6 μL | 10.8 μL |
| Sample | 4.8 μL | — |
| Total | 12 μL | 21.6 μL |

### Example 2 Detection of Multiple Barcodes

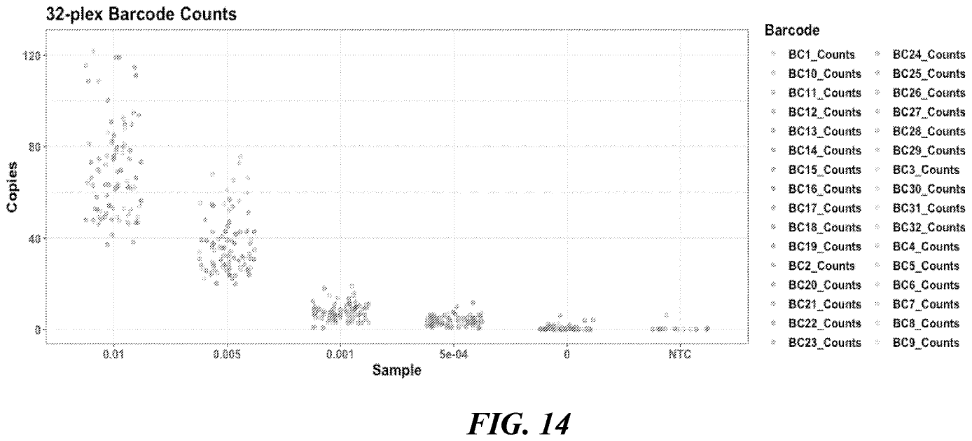

32 different nucleic acid barcode molecules are mixed together. Each different nucleic acid comprises (i) a universal forward primer binding region, (ii) a different probe binding region comprising different sets of universal probe binding sites (e.g., “barcode region”, and a (iii) universal reverse primer binding site. The barcode molecules can be detected using four channels and each barcode is coded such that each has a different combination of intensities that are generated in the four channels. The barcodes are diluted to 5%, 1%, 0.1%, and 0.01%, with 31 barcodes dilute in a mix of 1 barcode. This allows for a replicating a sample with a minor allele fraction (MAF). The PCR assay was set up comprising a universal forward primer, a universal reverse primers, and universal probes, and run and leveled using a AbsoluteQ instrument. The assay was run in triplicate. FIG. 14 shows data generated from the assay. Each dot represents a barcode, with the y-axis showing the copies detected and the x-axis demonstrating the dilution performed. All 32 barcodes were detected demonstrating the multiplexed detection of multiple different barcodes.

### Example 3 Molecular Inversion Probe-Like Oligonucleotide Detection Assay

FIG. 12 shows an example method using molecular inversion probe-like oligonucleotides. A sample comprising multiple targets is provided. Foreach analyte to be detected, a oligonucleotide is provided that has a first region that binds to the analyte, a second region that comprises one or more probe binding sites, and a third region that binds to the analyte at an adjacent region to the binding site of the first region. The oligonucleotides is allowed to hybridize to analytes in a sample. Following hybridization, a ligation or circularization reaction is performed that connects the two ends of the oligonucleotide. After the ligation reaction, excess linear nucleic acids are digested via an exonuclease reaction to remove unbound oligonucleotides and sample nucleic acids. The oligonucleotide can then be detected by the addition of universal probes and primers that bind to the oligonucleotides and generate signal associated with the probe binding sites. Based on barcoding or encoding of analytes to a specific barcode, the presence of an analytes can be determined.

### Example 4: Detection of Multiple Targets Using Tailed Primers

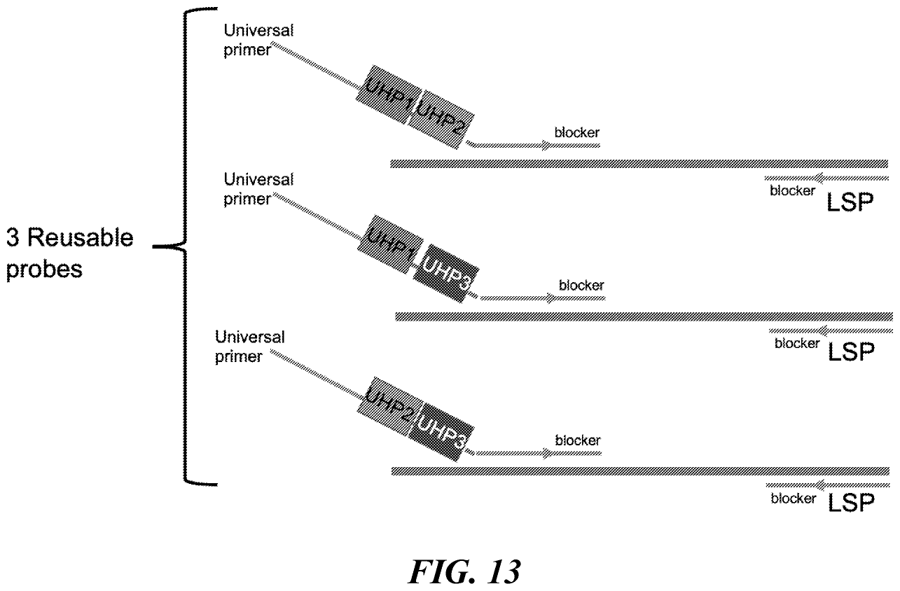

FIG. 13 shows an example schematic of an assay to detect multiple different loci and alleles. For each target, tailed primer is generated that has a region specific to the target, and a region of probe binding sites, and universal primer. For the three depicted loci, the first target has a primer that has a probe binding site of universal probe 1 (UHP1) and universal probe 2 (UHP2), the second target has a primer that has a probe binding site of universal probe 1 (UHP1) and universal probe 3 (UHP3), and the third target has a primer that has a probe binding site of universal probe 2 (UHP1) and universal probe 3 (UHP3). Each tailed primer is depicted with a blocker on the 3′ end that may be removed enzymatically by an enzyme that recognizes if a perfectly matched duplex is formed. Each tailed primer is depicted with universal primer site that can anneal to a primer. A mixture of the tailed primers and locus specific primers (“LSP”) are added. A enzyme to remove the blocker is added and a polymerase is added and an amplification reaction is allowed to occur, using the tailed primers and the LSPs. Universal probes that bind, the UHP1 site, UHP2 site and UHP3 site of the different primers. Another reaction is performed using a universal primer that binds to the universal primer site and a reverse primer (e.g. “LSP”). The probes cleaved via the polymerase reaction and the signals for each target are generated, with a signal of UHP1+UHP2 equating to the presence of target 1. UHP1+UHP3 equating to the presence of target 2, and. UHP2+UHP3 equating to the presence of target 3.

### Example 5: Detection of Multiple Targets in Mixed Samples

A sample was generated by making a cell line mixtures of NA12828 and NA12890. These cell lines were mixed such that a SNVs were present at various spiked percentages. This was done to replicate the detection of rare alleles or variants with a low MAF. 6 different targets were analyzed using oligonucleotides with probes site barcodes that were able to be detected in 4 color channels. The assays were run in triplicate.

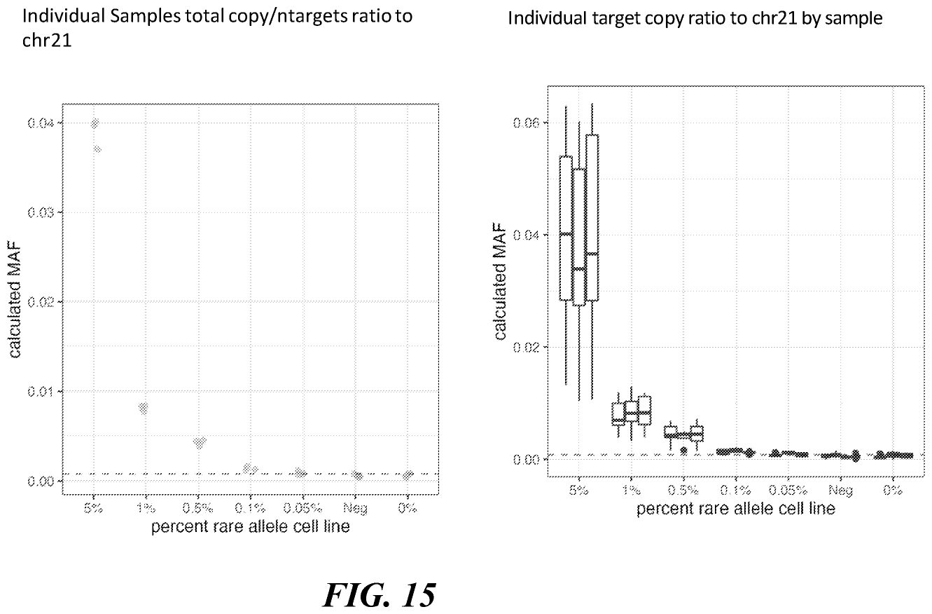

FIG. 15 shows data demonstrating detection of the targets and the calculated MAF, plotted against the percent of the rare allele cell line. The calculated MAF is similar to the expected spike in percentage for all spike in percentages. The left figure shows the MAF calculated for each sample, the total copies detected over the number of targets, normalized to chromosome 21. The right figure show the target copy ratio compared chromosome 21, for each sample.

Table 5 shows a summary of results for the above assays. For “position detected”, each number indicates the number of targets that were detected for each of the assays. For example, for 5% spike, 6 of the 6 positions were detected in first, second, and third assays. “True positive calls” indicates the number of assays that were successful at making a call out of the number of assays run. “False positive calls” indicates the number of assays that incorrectly called a control assay as a positive call out of the number of assays run.

TABLE 5
Summary of results for cell line mixture

| | Positions | True Positive | | |
|---|---|---|---|---|
| SNV Spike % | detected | Calls | Sensitivity | 95% CI |
| 5% | 6/6/6 | 3/3 | 100% | 29.2-100% |
| 1% | 6/6/6 | 3/3 | 100% | 29.2-100% |
| 0.5% | 6/6/6/ | 3/3 | 100% | 29.2-100% |
| 0.1% | 6/6/6 | 3/3 | 100% | 29.2-100% |
| 0.05% | 6/6/6 | 2/3 | 33% | 9.4-99.2% |
| | | False | | |
| | | Positive | | |
| | | calls | | |
| 0% | 2/3/0/0//1/3/3 | 0/7 | 100% | 59.0-100% |

Similar assays performed using different input nucleic acid mixtures. A mixture of synthetic cfDNA was used to generate similar rare alleles percentage, with 5 barcodes and targets used. Table 6 shows a summary of results for the synthetic cfDNA assay. For “position detected”, each number indicates the number of targets that were detected for each of the assays. For example, for 5% spike, 5 of the 5 positions were detected in first, second, and third assays. “True positive calls” indicates the number of assays that were successful at making a call out of the number of assays run. “False positive calls” indicates the number of assays that incorrectly called a control assay as a positive call out of the number of assays run. The table shows that the assay was able to successfully make calls at a sensitivity of 100% at SNV spike percentages as low as 0.1%, with not false positives.

TABLE 6
Summary of results for synthetic cfDNA

| | | True | | |
|---|---|---|---|---|
| SNV | Positions | Positive | | |
| Spike % | detected | Calls | Sensitivity | 95% CI |
| 5% | 5/5/5 | 3/3 | 100% | 29.2-100% |
| 1% | 5/5/5 | 3/3 | 100% | 29.2-100% |
| 0.5% | 5/5/5 | 3/3 | 100% | 29.2-100% |
| 0.1% | 2/3/3 | 3/3 | 100% | 29.2-100% |
| 0.05% | 2/3/2 | 1/3 | 33% | 0.8-90.6% |
| | | False | | |
| | | Positive | | |
| | | calls | | |
| 0% | 1/1/1/2/1/1/1/0/1/0 | 0/10 | | 69.1-100% |

A similar assay was also run using FFPE gDNA and a cell line gDNA background and 8 targets. Table 7 shows the results of the assay, demonstrating detection and differentiation of the two alleles at 0.2% SNV spike percentages.

TABLE 7
Summary of results for FFPE and cell line gDNA

| | Positions | True Positive | | |
|---|---|---|---|---|
| SNV Spike % | detected | Calls | Sensitivity | 95% CI |
| 50% | 8/8/8 | 3/3 | 100% | 29.2-100% |
| 6.7% | 7/7/7 | 3/3 | 100% | 29.2-100% |
| 2.4% | 7/6/7 | 3/3 | 100% | 29.2-100% |
| 1.1% | 7/6/6 | 3/3 | 100% | 29.2-100% |
| 0.5% | 6/6/5 | 3/3 | 100% | 29.2-100% |
| 0.2% | 3/4/3 | 3/3 | 100% | 29.2-100% |
| | | False Positive | | |
| | | calls | | |
| 0% | 2/1/3 | 0/3 | 100% | 29.2-100% |

### Example 6 MIP Based Detection Assays

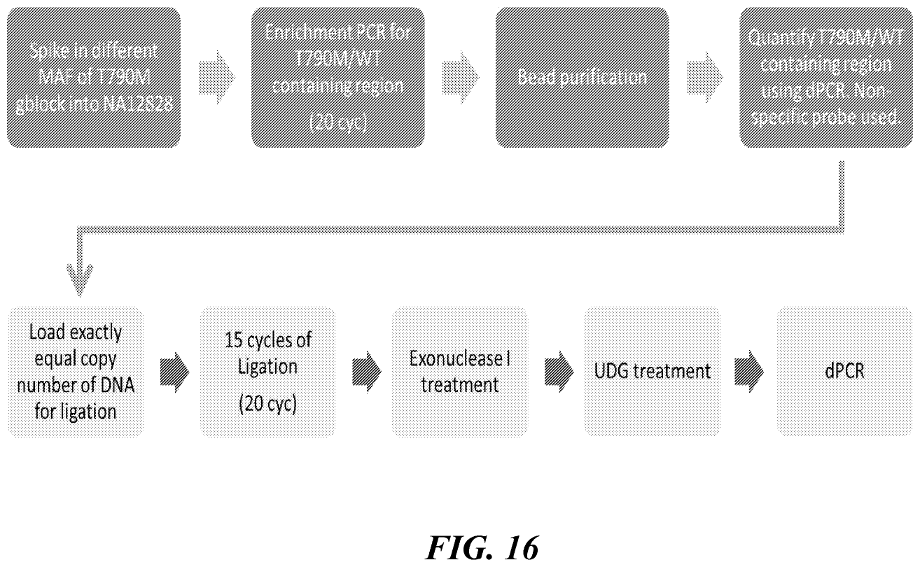

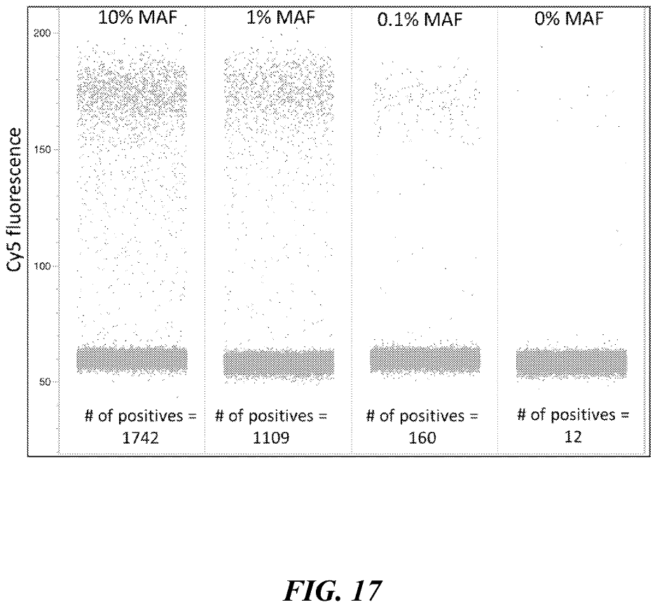

An assay for detection using molecular inversion probe like oligonucleotides was run to detect spike in of a SNV. FIG. 16 shows a schematic for the assay. Multiple samples were generated by spiking in a T790M EGFR gblock into a sample comprising a NA12828 cell line (wild type for T790M). Enrichment PCR was performed in the T790M/WT region and then purified using bead. The T790M/WT containing region was then quantified using dPCR and molecular inversion probe oligonucleotides of the disclosure. An equal copy number of molecular inversion probe oligonucleotides as loaded and then subjected to ligation to generate circularized probe molecules. The sample was then subjected to exonuclease treatment to remove linear nucleic acid, and then subjected to Uracil DNA-glycosylase to cleave the circularized probe molecules at a uracil and generate linearized nucleic acid. dPCR using non-specific probes (e.g., universal probes) was then performed to partition each linearized nucleic acid and detect the targets. FIG. 17 shows the results of the assay, demonstrating the detection and quantification of EGFR mutation, for spike in of 10% MAF, 1%, MAF, 0.1% MAF and 0% MAF. Each dot on the graph indicates a partition, with two population of dots of positive and negative partitions.

1. A method of identifying the presence of an analyte in a sample, the method comprising:
a) providing to said sample a primer oligonucleotide comprising a first region, wherein the first region hybridizes to said analyte and a second region comprising two or more distinct probe binding sites;
b) subjecting said primer oligonucleotide to an extension reaction, thereby generating a probe binding nucleic acid;
c) annealing (i) a second primer oligonucleotide and (ii) one or more probes to said probe binding nucleic acid;
d) subjecting said second primer oligonucleotide to an extension reaction thereby generating one or more signals; and
e) identifying the presence of said analyte based at least on detection of an intensity level and a wavelength of said one or more signals.

2. The method of claim 1, wherein said one or more signals is detected in more than one channel.

3. The method of claim 1, wherein said one or more signals is two or more signals.

4. The method of claim 1, wherein said one or more signals is detected in more than two channels.

5. The method of claim 1, wherein said one or more signals is detected in more than three channels.

6. The method of claim 1, wherein a signal of said one or more signals is detected at an intensity level of 1i.

7. The method of claim 1, wherein a signal of said one or more signals is detected at an intensity level of 2i.

8. The method of claim 1, wherein said one or more signals is generated from more than one probe that binds to said probe binding nucleic acid.

9. The method of claim 1, wherein said one or more signals is generated via degradation of one or more probes that binds to said probe binding nucleic acid.

10. The method of claim 1, wherein said second region comprises three or more distinct probe binding sites.

11. The method of claim 1, wherein said second region comprises four distinct probe binding sites.

12. The method of claim 1, prior to c), providing a first probe that binds to first probe binding site of said probe binding nucleic acid and a second probe that binds to a second probe binding site of said probe binding nucleic acid.

13. The method of claim 1, wherein a) further comprises providing a first reverse oligonucleotide that hybridizes to said analyte.

14. The method of claim 1, wherein b) comprises performing an amplification reaction to generate a plurality of amplicons of said probe binding nucleic acid.

15. The method of claim 1, further comprising, subsequent to a) and prior to b) generating a plurality of partitions.

16. The method of claim 1, wherein said first primer oligonucleotide further comprises a third region 5′ to said second and first region.

17. The method of claim 16, wherein c) comprises annealing said second primer oligonucleotide to said third region.

18. The method of claim 1, wherein c) comprises annealing said second primer oligonucleotide to at least a portion of said second region.

19. The method of claim 1, further comprising, identifying the presence of a second analyte, wherein a) comprises providing a third primer oligonucleotide comprising a first region that hybridizes to said second analyte and a second region comprising one or more probe binding sites.

20. The method of claim 1, wherein the analyte is detected at a sensitivity of a least 99%.

21-134. (canceled)
